(19) United States
(12) Patent Application Publication
(10) Pub. No.: US 2019/0142629 A1
(43) Pub. Date: May 16, 2019

(54) COOLING PACK AND THERAPEUTIC TOOL USED IN COOLING THERAPY

(71) Applicant: SHARP KABUSHIKI KAISHA, Sakai City, Osaka (JP)

(72) Inventors: DAISUKE SHINOZAKI, Sakai City (JP); YUKA UTSUMI, Sakai City (JP); HISANORI BESSHO, Sakai City (JP); HWISIM HWANG, Sakai City (JP); SATORU MOTONAMI, Sakai City (JP)

(21) Appl. No.: 16/096,417

(22) PCT Filed: Feb. 28, 2017
(86) PCT No.: PCT/JP2017/007864

§ 371 Date: Oct. 25, 2018

(30) Foreign Application Priority Data
Apr. 28, 2016 (JP) 2016-091593
Nov. 22, 2016 (JP) 2016-227282

(51) Int. Cl. A61F 7/10; A61F 7/02

(52) U.S. Cl. CPC A61F 7/10; A61F 2007/0036; A61F 2007/108; A61F 7/02

(57) ABSTRACT

A cooling pack is provided that can both maintain the human body at a suitable, low temperature and ensure a sufficient usage time. The present invention, in one aspect thereof, is directed to a cooling pack that cools a human body. The cold storage layer includes: a freezing medium having a phase transition temperature specified in a range of temperature suitable to maintain the human body at low temperature; and a cold-storage-layer-packaging member containing the freezing medium therein. The buffer layer includes: an antifreeze medium flexible at the phase transition temperature of the freezing medium; and a buffer-layer-packaging member made of a flexible material and containing the antifreeze medium therein. The buffer layer has a prescribed thermal conductivity, thermal diffusivity, thermal effusivity, and heat transmission coefficient. The buffer layer is brought into contact with human skin to transfer heat between the human body and the cold storage layer.

### TECHNICAL FIELD

The present invention relates to cooling packs that cool down a human body and also to therapeutic tools used in cooling therapy.

### BACKGROUND ART

Cooling therapies have been known such as icing and cryotherapy. Cooling therapy cools the entire human body or hot parts of the body, for example, by blowing cold air at the human body or by, as shown in FIG. 41, placing a cooling medium in contact with the skin of the human body. Patent Literature 1 discloses a cooling medium that is expected to provide increased comfort and fittedness and deliver sufficient cooling performance when worn on the human head. This cooling medium includes a plurality of horizontally coupled freezing media having a thickness of 15 to 35 mm and a non-freezing medium having a thickness of 5 to 15 mm. These freezing and non-freezing media are stacked and contained in an exterior bag.

### CITATION LIST

### Patent Literature

Patent Literature 1: Japanese Unexamined Patent Application Publication, Tokukaihei, No. 7-95998

### SUMMARY OF INVENTION

### Technical Problem

If a cooling medium is used to cool a diseased area in cooling therapy as shown in FIG. 41, the cooling medium may be too cold to the human body to use it for an extended period of time, failing to achieve a sufficient cooling time. Attempts have been made to address this issue, one of which is to provide, for example, a piece of cloth between the cooling medium and the human skin to maintain a suitable skin temperature. Although the cooling medium disclosed in Patent Literature 1 is intended for use on the human body, no attention is paid to its thermophysical properties and the temperature range it delivers while being used. If the cooling medium is placed in direct contact with the skin, the cooling medium will remove too much heat from the human body.

Humans have a nerve called TRPA1, which perceives a skin temperature at or below 17° C. as a pain. For this reason, thorough consideration should be given to skin temperature when cooling the human body, to avoid stimulating this nerve. A cooling medium that cools skin temperature to or below 17° C. is difficult to use properly for an extended period of time and may in some cases cause frostbite. Patent Literature 1 discloses a cooling medium that is assumed to be used on the human body, but gives no description of, for example, the thermophysical properties of the material used as the cooling medium or of the temperature range of the freezing medium. Use of this cooling medium may therefore stimulate TRPA1. The cooling medium is not appropriate to wear for an extended period of time.

In addition, purposes and measures vastly differ between those cases where first aid is performed for inflammation in an acute trauma and those cases where rehabilitation or palliative care is given. No cooling packs have been proposed that address these differences.

The present invention, made in view of these issues, has an object to provide a cooling pack that both maintains the human body at a suitable, low temperature and ensures a sufficient usage time and also to provide a therapeutic tool used in cooling therapy.

### Solution to Problem

To achieve the object, the present invention, in one aspect thereof, is directed to a cooling pack that cools a human body, the cooling pack including: a freezing medium having a phase transition temperature specified in a range of temperature, the range being suitable to maintain the human body at low temperature; and a first container section containing the freezing medium therein, wherein the freezing medium and the first container section constitute a cold storage layer. The cooling pack may further include: an antifreeze medium flexible at the phase transition temperature of the freezing medium; and a second container section including a flexible material and containing the antifreeze medium therein, wherein: the antifreeze medium and the second container section constitute a buffer layer having a prescribed thermal conductivity, a prescribed thermal diffusivity, a prescribed thermal effusivity, and a prescribed heat transmission coefficient; and the buffer layer transfers heat between the human body and the cold storage layer when brought into contact with skin of the human body.

### Advantageous Effects of Invention

The present invention, in one aspect thereof, can both maintain the human body at a suitable, low temperature and ensure a sufficient usage time.

### BRIEF DESCRIPTION OF DRAWINGS

FIG. 1 is a concept diagram of a cooling pack in accordance with the present embodiment.

FIG. 2 is a table of therapeutic purposes, diseased areas, skin temperatures, and cooling medium usage times in a cooling therapy.

FIG. 3 is a diagram of activation temperature thresholds for a temperature-sensitive human TRPA channel.

FIG. 4A is a schematic diagram of a cooling pack in accordance with Example 1.

FIG. 4B is a schematic diagram of a cooling pack in accordance with Example 1.

FIG. 5 is a table of values of thermophysical properties.

FIG. 6A is a diagram of a variation example of a cold storage layer in accordance with Example 1.

FIG. 6B is a diagram of a variation example of a cold storage layer in accordance with Example 1.

FIG. 6C is a diagram showing a comparison of a cold storage layer of a single-piece structure and a cold storage layer of a multipiece-coupled structure.

FIG. 7A is a diagram illustrating a function of a buffer layer.

FIG. 7B is a diagram illustrating a function of a buffer layer.

FIG. 7C is a schematic diagram of buffer layers and cold storage layers both having an articulation mechanism.

FIG. 7D is a cross-sectional view of the condition of a cold storage layer and a buffer layer when a cooling pack with no articulation mechanisms is placed along the length of the paper on which FIG. 7D is drawn.

FIG. 7E is a cross-sectional view of the condition of cold storage layers and buffer layers when a cooling pack with articulation mechanisms is placed along the length of the paper on which FIG. 7E is drawn.

FIG. 8 is a table of values of thermophysical properties of a buffer layer.

FIG. 9 is a diagram representing a method of calculating the thermal conductivity of a multilayer body.

FIG. 10A is a diagram illustrating how a pseudo-skin temperature measurement experiment is conducted using a thermal manikin.

FIG. 10B is a diagram illustrating how a pseudo-skin temperature measurement experiment is conducted using a thermal manikin.

FIG. 10C is a diagram illustrating how a pseudo-skin temperature measurement experiment is conducted using a thermal manikin.

FIG. 11A is a set of diagrams (contour figures) showing results (measurements) of a pseudo-skin temperature measurement experiment using a thermal manikin.

FIG. 11B is a line map version of FIG. 11A.

FIG. 12 is a graph representing a relationship between elapsed time and surface temperature.

FIG. 13 is an illustration of the condition of a cooling pack prepared using a supporter and worn around the arm.

FIG. 14 is an example questionnaire in which the level of comfort/discomfort is marked on a scale every time a unit time has elapsed.

FIG. 15 is a diagram of three types of buffer layer samples, including schematics of their structures.

FIG. 16 is a diagram representing results of sensory evaluations.

FIG. 17 is a table of the structures of buffer layer samples and the compositions and q-max values of packaging members.

FIG. 18A is a schematic diagram of a structure of a cooling pack in accordance with Example 2.

FIG. 18B is a schematic diagram of a structure of a cooling pack in accordance with Example 2.

FIG. 19A is a schematic diagram of a structure of a cooling pack in accordance with Example 3.

FIG. 19B is a schematic diagram of a structure of a cooling pack in accordance with Example 3.

FIG. 20A is a diagram of a process of manufacturing a cooling pack in accordance with Example 3.

FIG. 20B is a diagram of a process of manufacturing a cooling pack in accordance with Example 3.

FIG. 21 is a diagram of a cooling pack in accordance with Example 3 prepared as a pack-in-pack model.

FIG. 22A is a schematic diagram of a structure of a cooling pack and a therapeutic tool in accordance with Example 4.

FIG. 22B is a schematic diagram of a structure of a cooling pack and a therapeutic tool in accordance with Example 4.

FIG. 22C is an illustration of a usage example.

FIG. 23 is a diagram representing a relationship between the in-plane lengths of a cold storage layer and a buffer layer.

FIG. 24 is a diagram showing a thermochromic medium being applied to cold storage layers.

FIG. 25 is a diagram listing preferred thermochromic media for cooling packs in accordance with the present embodiment.

FIG. 26A is a schematic diagram of a structure of a cooling pack in accordance with Example 5.

FIG. 26B is a schematic diagram of a structure of a cooling pack in accordance with Example 5.

FIG. 27A is a set of diagrams (contour figures) showing results (measurements) of a pseudo-skin temperature measurement experiment using a thermal manikin.

FIG. 27B is a line map version of FIG. 27A.

FIG. 28 is a schematic diagram of a structure of a cooling pack in accordance with Example 6.

FIG. 29 is a schematic illustration of an automatic packing machine for manufacturing a cooling pack in accordance with Example 6.

FIG. 30 is a diagram representing a process of manufacturing a cooling pack 60 in a vertical form-fill seal machine.

FIG. 31 is a diagram showing a comparison of a cooling pack of a vertical pillow type and a cooling pack of a four-corner-sealed type, the former being in accordance with Example 6.

FIG. 32A is a set of diagrams (contour figures) showing results (measurement) of a pseudo-skin temperature measurement experiment using a thermal manikin.

FIG. 32B is a line map version of FIG. 32A.

FIG. 33 is an illustration of a cooling pack for palm cooling in accordance with Example 7.

FIG. 34 is an illustration of a cooling pack for palm cooling in accordance with Example 8.

FIG. 35 is an illustration of a cooling pack for palm cooling in accordance with Example 9.

FIG. 36 is an illustration of a cooling pack for palm cooling in accordance with Example 10.

FIG. 37 is an illustration of a cooling pack for palm cooling in accordance with Example 11.

FIG. 38A is an illustration of a cooling pack for palm cooling in accordance with Example 12.

FIG. 38B is an illustration of a cross-section model of a blood vessel.

FIG. 39A is an illustration of a cooling pack in accordance with Example 13.

FIG. 39B is an illustration of a cooling pack in accordance with Example 14.

FIG. 39C is an illustration of a cooling pack in accordance with Example 15.

FIG. 40 is an illustration of a cooling pack in accordance with Example 16.

FIG. 41 is a diagram of an example state of use of a conventional cooling medium.

### DESCRIPTION OF EMBODIMENTS

Conventional cooling media are too cold to the human body to cool a diseased area and are hardly usable for an extended period of time, making it impossible to achieve a sufficient cooling time. Paying attention to these facts, the inventors of the present invention have discovered that suitable skin temperature and sufficient usage time can be achieved by stacking a freezing medium that changes phase at a specific temperature in a range of temperature, the range being suitable to maintain the human body at low temperature, and an antifreeze medium that, without freezing, remains flexible at that phase transition temperature of the freezing medium and further by specifying the thermal conductivity, thermal diffusivity, thermal effusivity, and heat transmission coefficient of a buffer layer that is brought into direct contact with the human body, which has led to the present invention.

Specifically, the present invention, in one aspect thereof, is directed to a cooling pack that cools a human body, the cooling pack including: a freezing medium having a phase transition temperature specified in a range of temperature, the range being suitable to maintain the human body at low temperature; and a first container section containing the freezing medium therein, wherein the freezing medium and the first container section constitute a cold storage layer. The cooling pack may further include: an antifreeze medium flexible at the phase transition temperature of the freezing medium; and a second container section including a flexible material and containing the antifreeze medium therein, wherein: the antifreeze medium and the second container section constitute a buffer layer having a prescribed thermal conductivity, a prescribed thermal diffusivity, a prescribed thermal effusivity, and a prescribed heat transmission coefficient; and the buffer layer transfers heat between the human body and the cold storage layer when brought into contact with skin of the human body.

The inventors of the present invention have thus succeeded in both maintaining the human body at a suitable, low temperature and ensuring a sufficient usage time. The following will specifically describe embodiments of the present invention in reference to drawings.

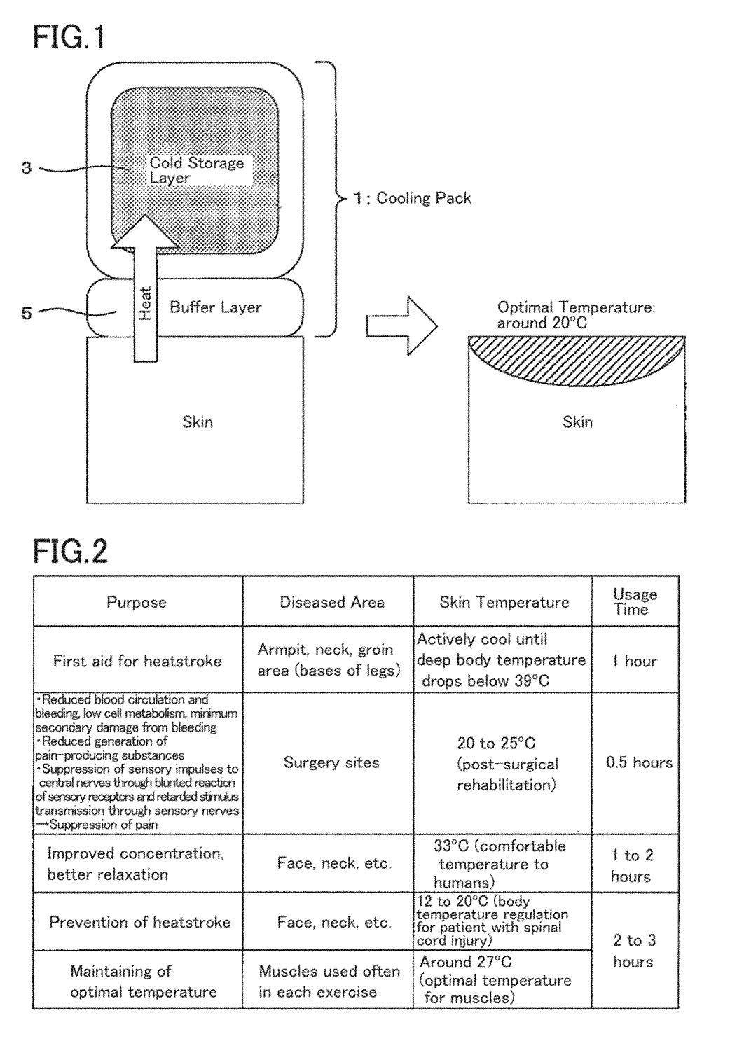

FIG. 1 is a concept diagram of a cooling pack in accordance with the present embodiment. A cooling pack 1 includes a cold storage layer 3 and a buffer layer 5. The cold storage layer 3 has prescribed thermal properties and to put it in more specific terms, contains a freezing medium that has a phase transition temperature of 12° C. The buffer layer 5 has prescribed thermal and mechanical properties and contains an antifreeze medium that is in liquid phase at 12° C. This structure maintains skin temperature at around 20° C. when the cooling pack 1 in accordance with the present embodiment is brought into direct contact with human skin.

FIG. 2 is a table of therapeutic purposes, diseased areas, skin temperatures, and cooling medium usage times in cooling therapy. The cooling medium is placed under the armpit, around the neck, or on the groin area of the human body as a first aid for heatstroke or like disorders. In this procedure, the skin is actively cooled until deep body temperature drops below 39° C. The cooling medium is used for an hour for this purpose.

In cooling for the purpose of reduced blood circulation and bleeding, one is expected to lower cell metabolism and thereby reduce ischemia-induced secondary damage to a minimum. Cooling is also done to reduce the generation of pain-producing substances and to suppress sensory impulses to the central nerve system through blunted reaction of sensory receptors and retarded stimulus transmission through sensory nerves. For these purposes, the cooling medium is placed on surgery sites for half an hour, and the skin temperature is 20° C. to 25° C.

Cooling has further purposes including improved mental concentration and better relaxation. For these purposes, the cooling medium is placed, for example, on the forehead or neck for 1 to 2 hours, and the skin temperature is, for example, 33° C., which is a comfortable temperature to humans. To prevent heatstroke, the cooling medium is placed, for example, on the forehead or neck for 2 to 3 hours, and the skin temperature is, for example, 12 to 20° C. in the case of body temperature regulation for a patient with spinal cord injury. In cooling for the purpose of maintaining an optimal temperature, the cooling medium is placed for 2 to 3 hours on muscles used often in each exercise, and the skin temperature is around 27° C., which is an optimal temperature for muscles.

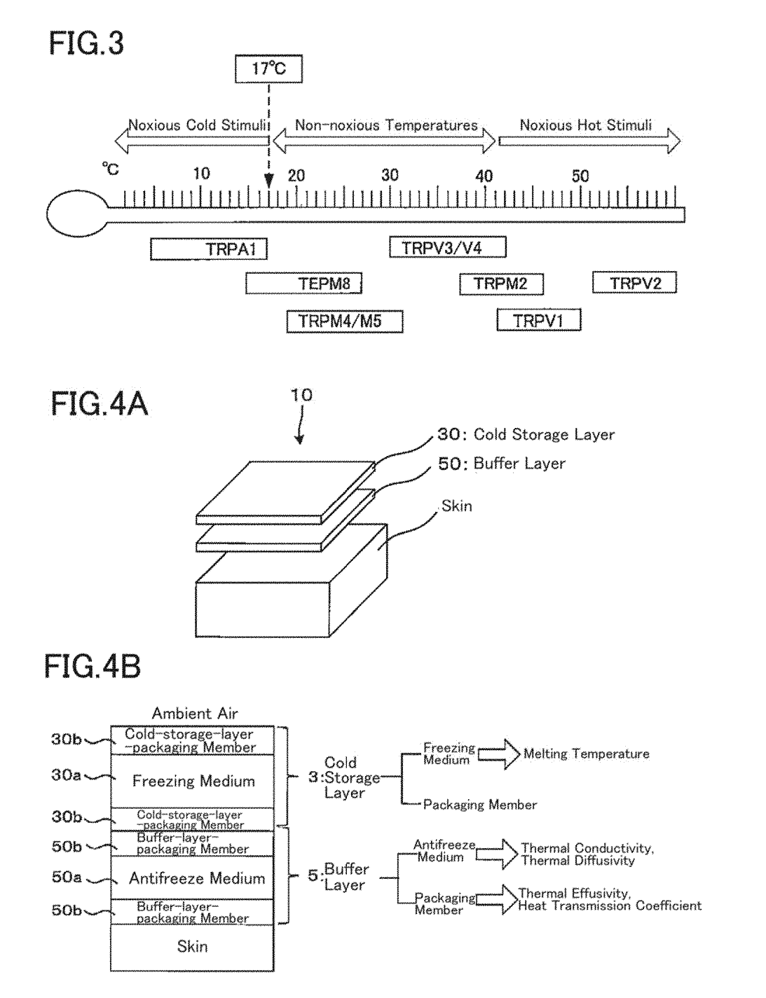

FIG. 3 is a diagram of activation temperature thresholds for a temperature-sensitive human TRPA channel. Pain occurs in the human body because a stimulus applied to a nociceptor located on a nerve ending is transferred to the brain. Such a nerve ending receptor organ includes receptors for various stimuli collectively called TRP receptors. These receptors send or do not send signals for different temperature ranges. A cold stimulus at or below 17° C. activates the TRPA1 channel as shown in FIG. 3. In other words, humans recognize a stimulus causing a 17° C. or lower skin temperature as a pain. Therefore, the skin temperature should be maintained at no lower than 17° C. in cooling over an extended period of time.

### EXAMPLE 1

FIGS. 4A and 4B are schematic diagrams of a cooling pack in accordance with Example 1. This cooling pack 10 includes a cold storage layer 30 and a buffer layer 50. The cold storage layer 30 is prepared by encasing, in a cold-storage-layer-packaging member 30b, a freezing medium 30a that freezes at a specific temperature. The cold-storage-layer-packaging member 30b constitutes a first container section. Meanwhile, the buffer layer 50 is prepared by encasing, in a buffer-layer-packaging member 50b, an antifreeze medium 50a that does not freeze at the freezing temperature of the freezing medium 30a in the cold storage layer 30. The buffer-layer-packaging member 50b is made of a flexible material and constitutes a second container section. The buffer layer 50 has a function of reducing the heat removed from human skin by the cold storage layer 30 and a function of improving attachment to the skin.

In the cooling pack 1 in accordance with Example 1, the functions of the cold storage layer 30 and the buffer layer 50 are specified as in the following in order to both prevent the skin temperature of the human body from dropping below 17° C. while in use and maintain the skin temperature at around 20° C. Specifically, the cold storage layer 30 has a melting temperature of 12° C. The buffer layer 50 has a thermal conductivity of 0.589, a thermal diffusivity of 1.589×10⁻⁷, a thermal effusivity of 1,502, and a heat transmission coefficient of 115. These thermophysical properties do not necessarily have such values and may have a range of values in the present invention. Specifically, the thermal conductivity may range from 0.584 to 0.590, the thermal diffusivity from 1.503×10⁻⁷ to 1.537×10⁻⁷, the thermal effusivity from 1,495 to 1,503, and the heat transmission coefficient from 113 to 115.

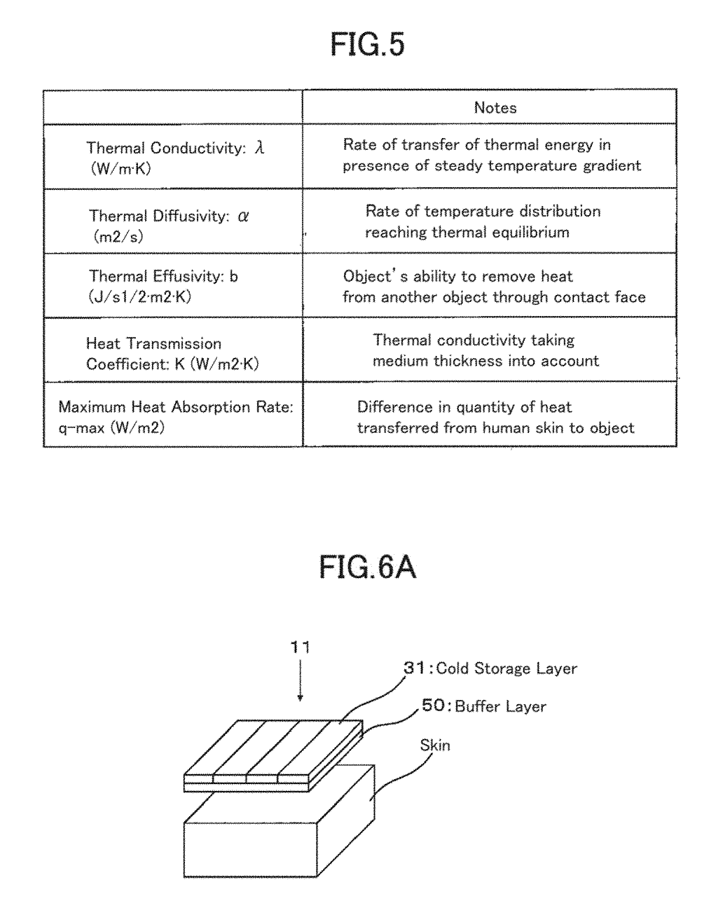

FIG. 5 is a table of values of thermophysical properties. Thermal conductivity, or λ, (W/m·K) is a rate of transfer of thermal energy in the presence of a steady temperature gradient. Thermal diffusivity, or α, (m²/s) is a rate of a non-uniform temperature distribution reaching a thermal equilibrium. Thermal effusivity, b, (J/s^{1/2}·m²·K) indicates an object's ability to remove heat from another object through a contact face. Heat transmission coefficient, or K, (W/m²·K) is a thermal conductivity that takes the thickness of the medium into account. Maximum heat absorption rate, or q-max, (W/m²), which is detailed below, indicates a difference in the quantity of heat transferred from human skin to an object.

A description is now given of maximum heat absorption rate, or q-max, (W/m²). It is known that a human can feel differently upon touching different objects of the same temperature. For example, iron and other like metals feel cold, whereas wool and other like fibers feel warm. These feelings on the skin are termed “contact cold feelings,” and the maximum heat absorption rate, q-max, is used as an index. The maximum heat absorption rate, q-max, has a greater value if the object is evaluated to be colder and a smaller value if the object is evaluated to be warmer. In other words, one will feel more comfortable upon their skin touching art object if the stimulus is smaller (if less heat is transferred).

### Freezing Medium

Example 1 uses, as a freezing medium, a material obtained by dissolving tetrabutylammonium bromide (hereinafter, “TBAB”) in water to 40 wt %. The present invention is by no means limited to this concentration. Additionally, an additive may be added to restrain supercooling. Examples include, but are by no means limited to, sodium tetraborate hydrates (pentahydrate and decahydrate), disodium hydrogen phosphate, and sodium carbonate. For the cooling pack in accordance with Example 1, “TBAB_40 wt %+sodium tetraborate_2%” or “TBAB_40 wt %+disodium hydrogen phosphate_3%+sodium carbonate_2%” is used. This aqueous solution is frozen for use as a freezing medium. Because this freezing medium has a phase transition temperature of 12° C., the freezing medium remains at 12° C. when it melts (changes from frozen, solid phase to a partially solid, partially liquid phase).

### Variation Example of Cold Storage Layer

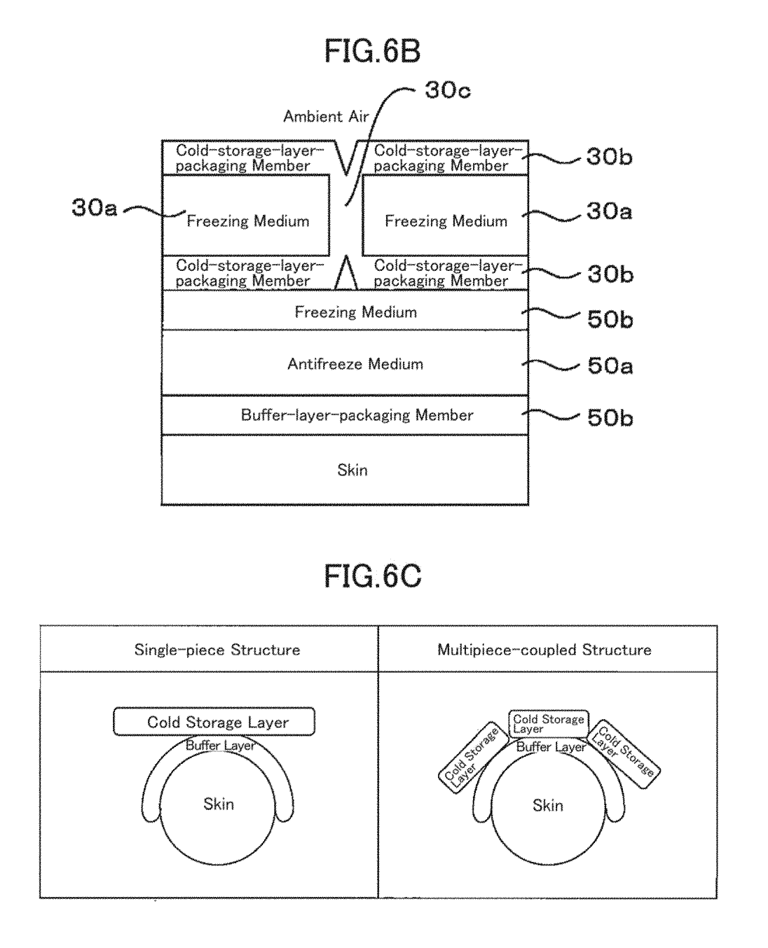

FIGS. 6A and 6B are diagrams of a variation example of a cold storage layer in accordance with Example 1. In this variation example, there is provided a plurality of cold storage layers 31 coupled together by articulation mechanisms 30c. Each cold storage layer 31 includes a freezing medium 30a and a cold-storage-layer-packaging member 30b encasing the freezing medium 30a. FIG. 6C shows on the left side thereof a single-piece structure with no articulation mechanisms being worn, for example, around a human arm or leg. The single-piece structure, being frozen and therefore inflexible, can only touch a point on the arm or leg. Meanwhile, as shown on the right side of FIG. 6C, the cold storage layers 31, coupled by the articulation mechanisms and therefore better fitting the human body, are capable of uniformly cooling a larger area.

### Buffer Layer

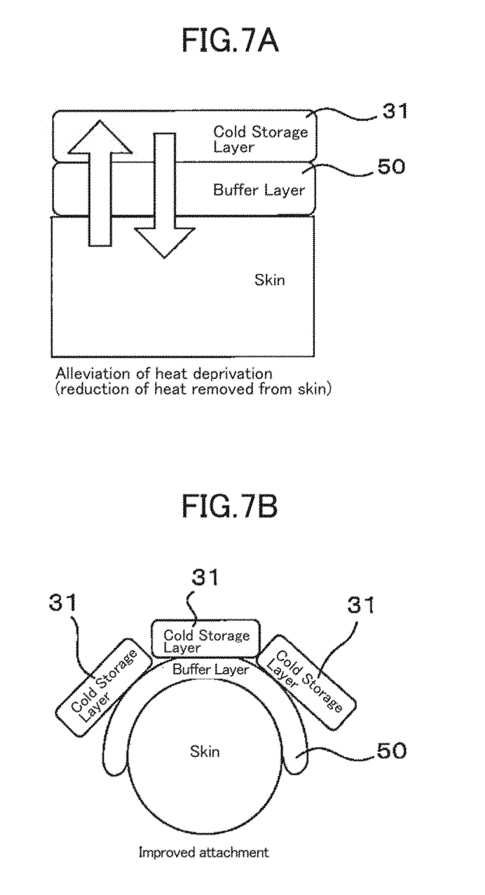

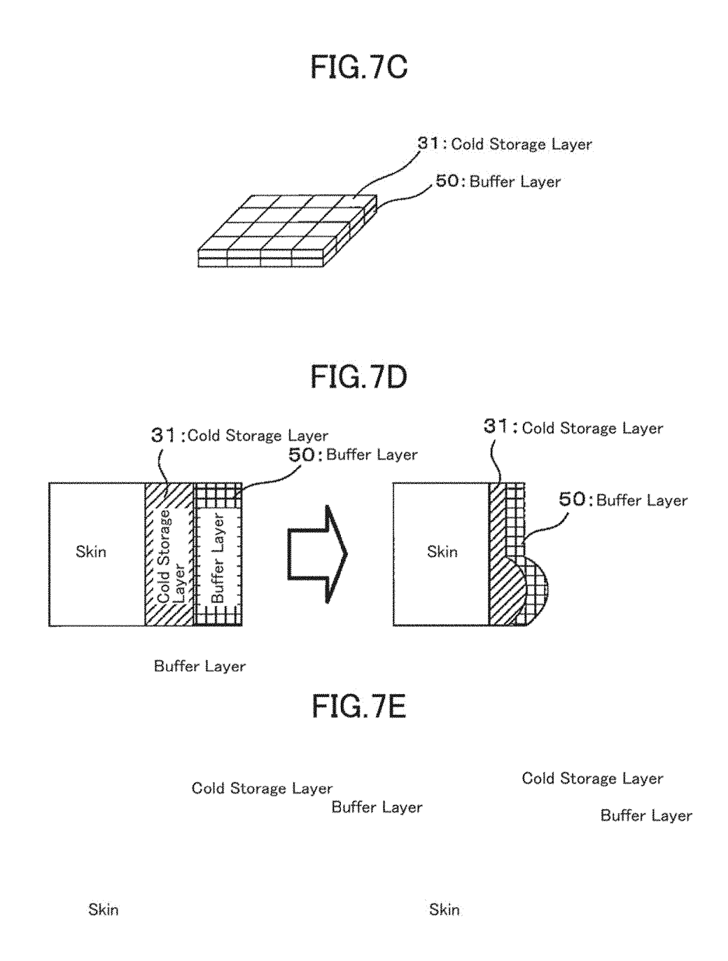

FIGS. 7A and 7B are diagrams illustrating a function of a buffer layer. Referring to FIG. 7A, the buffer layer 50 is brought into contact with the skin of the human body to transfer heat between the human body and the cold storage layer(s) 30 or 31. This function allows for alleviation of heat deprivation (reduction of heat removed from the skin). Since the antifreeze medium 50a in the buffer layer 50 is in liquid phase at the phase transition temperature of the freezing medium 30a, and the buffer-layer-packaging member 50b is flexible, the buffer layer 50 attaches well to the skin as shown in FIG. 7B. There may be provided a plurality of buffer layers 50 coupled together by articulation mechanisms as shown in FIG. 7C. In this alternative structure, the buffer layers 50 may be coupled in only one of the length and width directions or in both of these two directions. Likewise, there may be provided a plurality of cold storage layers coupled by articulation mechanisms. Referring to FIG. 7D, if a cooling pack is placed along the length of the paper on which FIG. 7D is drawn, and the temperature of the cold storage layers 31 and the buffer layer 50 rises during use, the aqueous solution in each layer collects in the vertical direction. As a result, there occur heat capacity differences in the cooling pack, which causes “non-uniform cooling.” FIG. 7E shows a structure in which there is provided a plurality of cold storage layers coupled together by articulation mechanisms and a plurality of buffer layers coupled together by articulation mechanisms. The phenomenon illustrated in FIG. 7C may occur in each layer, but this structure suppresses potential heat capacity differences, thereby enabling prevention of non-uniform cooling.

### Antifreeze Medium

The antifreeze medium 50a in Example 1 may be obtained by dissolving, for example, sodium chloride (NaCl) or potassium chloride (KCl) in water. These are not the only examples, and any material may be used that does not freeze at the temperature at which the freezing medium 30a freezes. Since the freezing medium 30a in the cold storage layers 31 is used in solid state, the freezing medium 30a, even in the multipiece-coupled structure described in the variation example above, practically touches a point on the skin of the human body. If the buffer layer 50 includes an antifreeze medium 50a that remains fluid at the phase transition temperature of the freezing medium 30a, the buffer layer 50 attaches well to the skin, which enables uniform cooling. In this situation, the viscosity of the antifreeze medium 50a may be increased to make it easier to keep the cold storage layers 31 bent. As discussed here, the provision of the buffer layer 50 prevents the frozen cold storage layers 31 from coming in direct contact with the skin, thereby protecting the skin from rapid heat removal.

Next, it will be described how thermophysical property values are specified for a buffer layer. As mentioned earlier, the buffer layer 50 is constructed by encasing the antifreeze medium 50a, which is an aqueous solution of NaCl mixed with a thickening agent, in the buffer-layer-packaging member 50b. The antifreeze medium 50a and the buffer-layer-packaging member 50b are, however, regarded as a single physical object made of a single substance for the sake of determining their thermophysical property values.

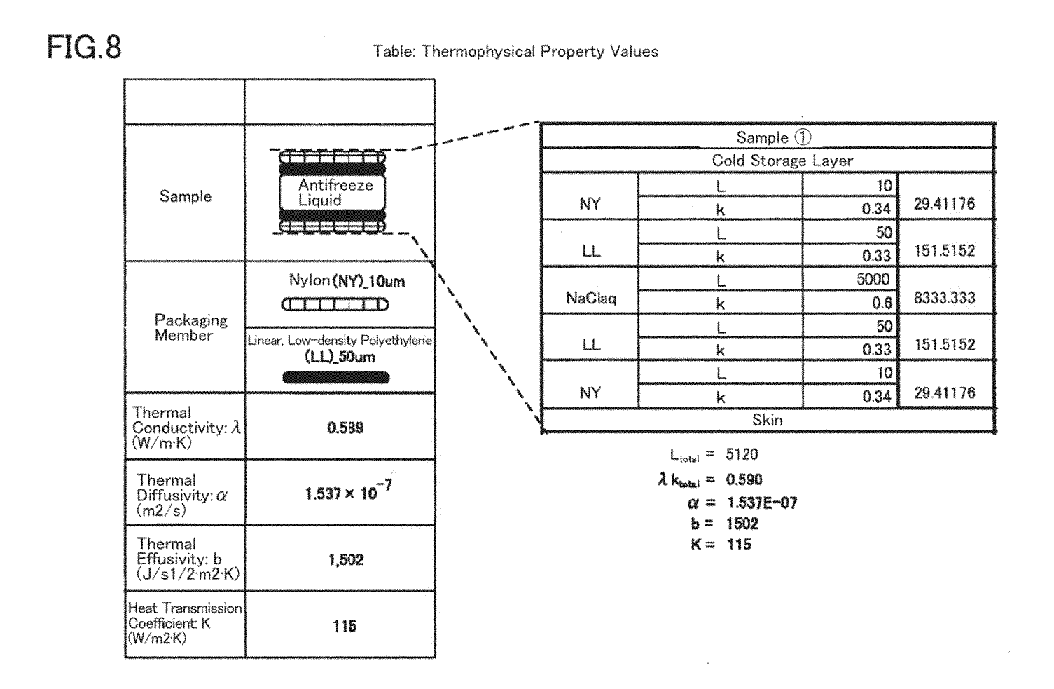

FIG. 8 is a table of values of thermophysical properties of a buffer layer. Letting λ denote thermal conductivity in W/m·K, α thermal diffusivity in m²/s, b thermal effusivity in J/s^{1/2}·m²·K, and K heat transmission coefficient in W/m²·K and also letting ρ denote density in kg·m³ and c specific heat capacity in J·kg/K, the following equations hold:

λ=α×ρ×c,

α=λ.ρ×c, and

b=√λ×ρ×c.

The density and specific heat of the buffer layer are regarded here as being equal to those of the aqueous solution of NaCl and assumed to have the following values:

Specific Heat c=3,337 J/kg/K, and

Density ρ=1,147.8 kg/m³.

These values are taken from pages 161 and 162 of “Thermophysical Property Handbook, New Edition” edited by Japan Society of Thermophysical Properties.

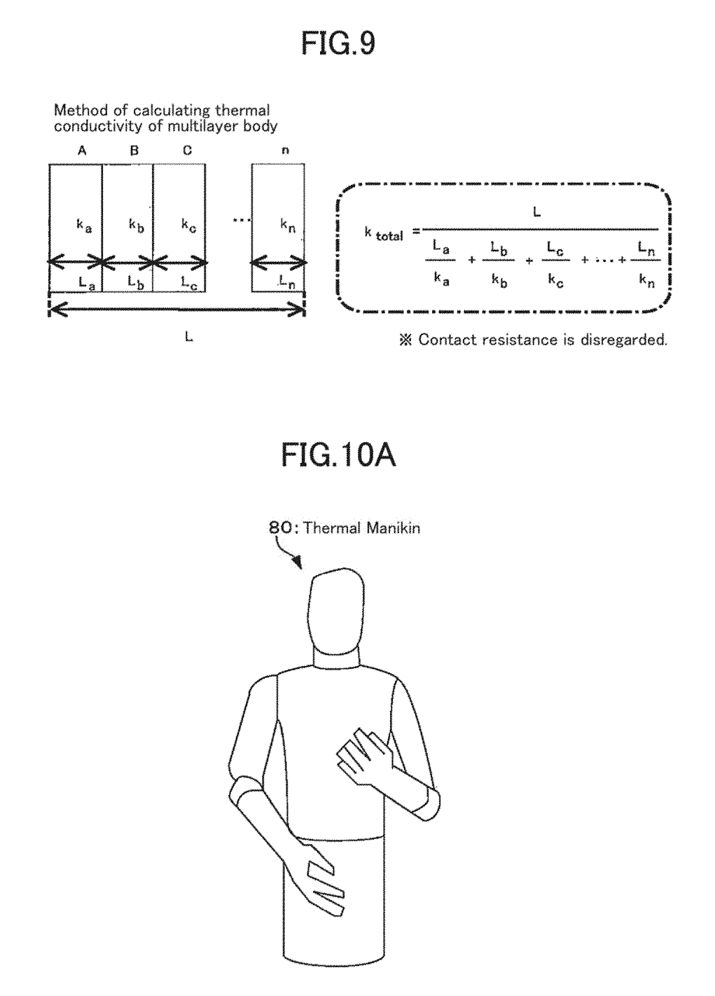

FIG. 9 is a diagram representing a method of calculating the thermal conductivity of a multilayer body. Contact resistance is disregarded here.

The values of thermophysical properties of the buffer layer were specified in this manner. Specific values are given in FIG. 8: the buffer layer has a thermal conductivity of 0.589, a thermal diffusivity of 1.537×10⁻⁷, a thermal effusivity of 1,502, and a heat transmission coefficient of 115.

The antifreeze medium may have an alternative composition as shown in the table below, depending on its usage. If the freezing medium in the cold storage layer is to be frozen in a refrigerator (at around 4° C.), the antifreeze medium in the buffer layer may be composed of water alone, a combination of water and a thickening agent, a combination of water, sodium chloride, and a thickening agent, or a combination of water, potassium chloride, and a thickening agent. Since the internal temperature of a refrigerator does not fall below 0° C., the antifreeze medium in the buffer layer, even if composed solely of water, does not freeze. Meanwhile, if the freezing medium is to be frozen quickly in a freezer, the antifreeze medium may be composed of a combination of water, sodium chloride, and a thickening agent or a combination of water, potassium chloride, and a thickening agent. The freezing medium in the cold storage layer can be frozen more quickly in a freezer (the internal temperature ranges from −18 to 20° C.) than in a refrigerator. Since the internal temperature of a freezer falls below 0° C., the buffer layer, if composed solely of water, will freeze. This is prevented by adding, for example, sodium chloride or potassium chloride.

TABLE 1

| Situation | Frozen Only in Refrigerator | Frozen in Freezer |
|---|---|---|
| Condition | Normal Freezing (in | Quick Freezing (in Freezer) |
| | Refrigerator) | |
| Composition of | Water | Water, Sodium Chloride, |
| Antifreeze | Water and Thickening | and Thickening Agent |
| Medium in | Agent | Water, Potassium |
| Buffer Layer | Water, Sodium Chloride, | Chloride, and Thickening |
| | and Thickening Agent | Agent |
| | Water, Potassium | |
| | Chloride, and Thickening | |
| | Agent | |

### Skin Temperature Measurement Experiment

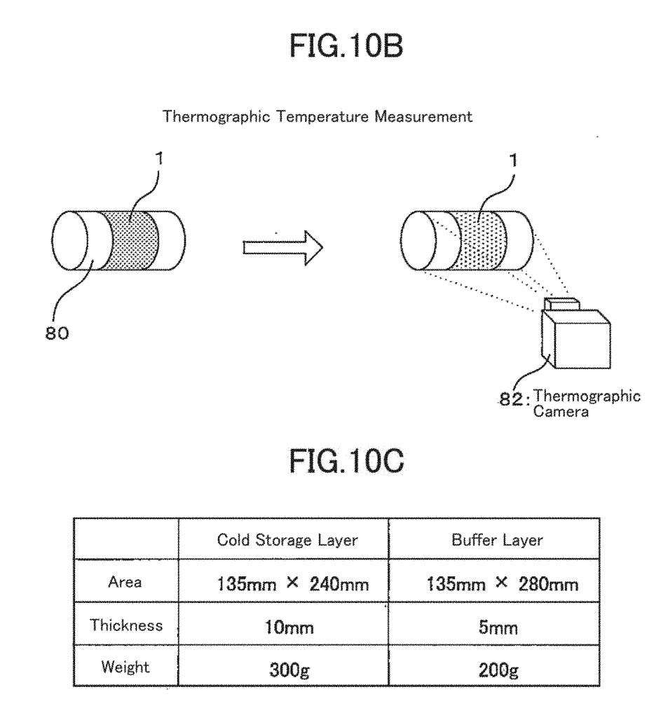

FIGS. 10A, 10B, and 10C are diagrams illustrating how a pseudo-skin temperature measurement experiment is conducted using a thermal manikin. A thermal manikin 80 has a function of artificially reproducing the generation of heat by a human body at ambient temperature. A cooling pack 1 was prepared in accordance with Example 1 for experimental purposes and attached to the thermal manikin 80. Changes in the temperature of the artificial skin surface were measured by a thermographic camera 82. In the cooling pack 1, the cold storage layers 31 were TBAB, and the buffer layer 50 was an aqueous solution of NaCl. In this experiment, a surface temperature distribution was obtained from the surface of the thermal manikin 80 (≈skin surface) immediately after the cooling pack 1 was removed.

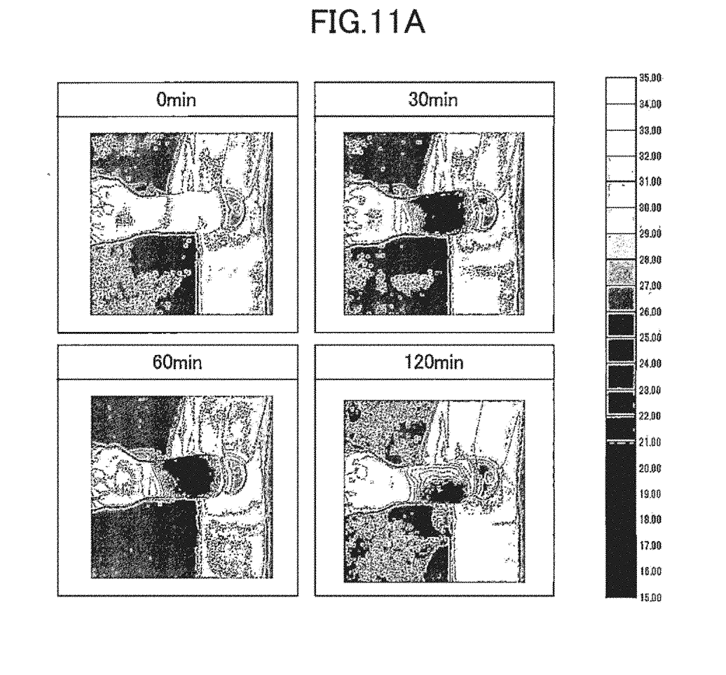

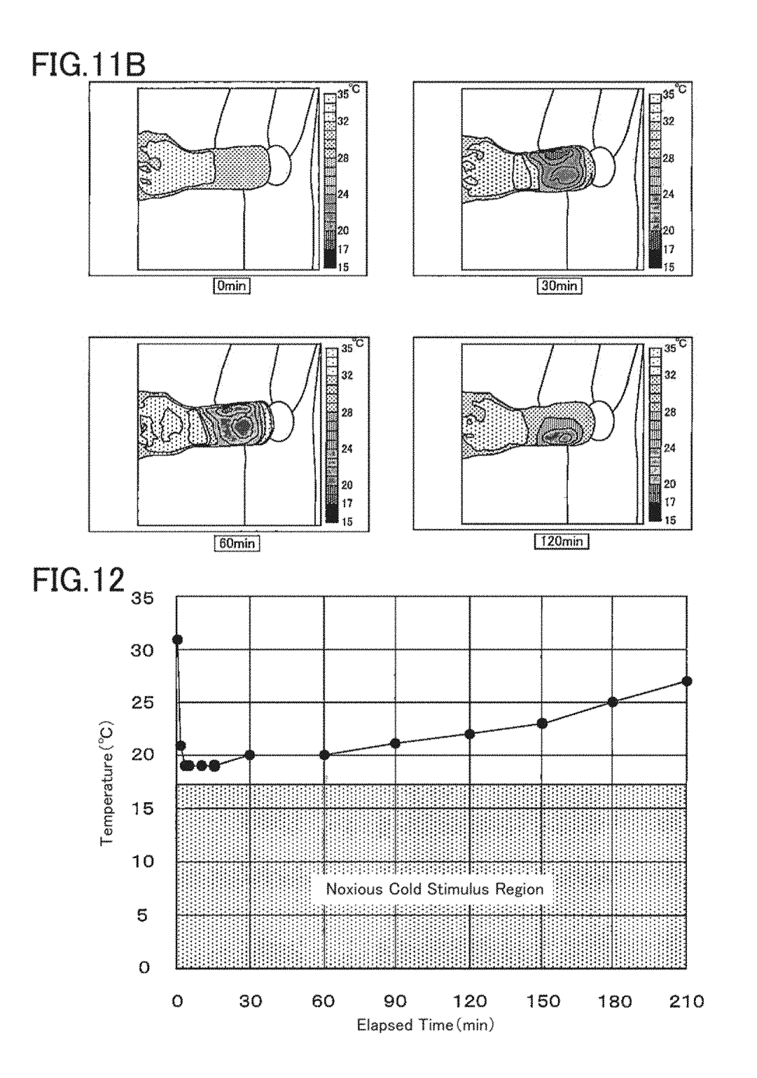

FIG. 11A is a set of diagrams (contour figures) showing results (measurements) of a pseudo-skin temperature measurement experiment using a thermal manikin. FIG. 11B is a line map version of FIG. 11A. FIGS. 11A and 11B selectively show measurements taken 0, 30, 60, and 120 minutes after the measurement was started. FIG. 12 is a graph representing a relationship between elapsed time and surface temperature. FIGS. 11 and 12 demonstrate that the skin temperature does not go into the noxious cold stimulus region and is maintained at around 20° C., which is suitable to the human body, for 120 minutes. It is therefore concluded that the use of a buffer layer having a set of thermophysical properties specified as above and a cold storage layer made of TBAB, which has a melting temperature of 12° C., enables the skin temperature to be maintained within a range of temperatures that is not painful to humans for an extended period of time.

### Sensory Evaluation

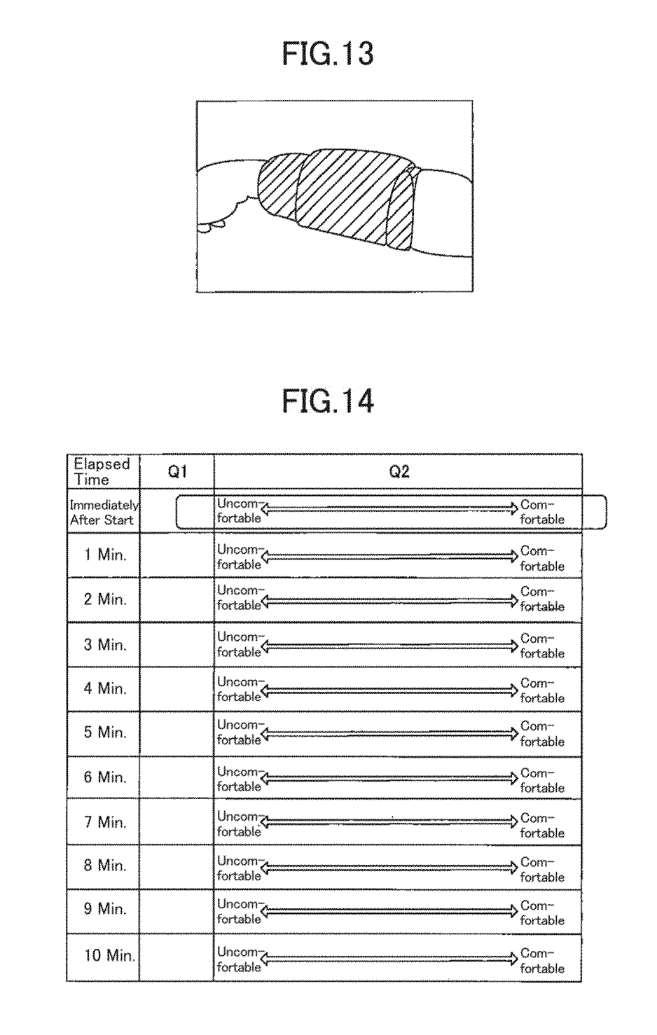

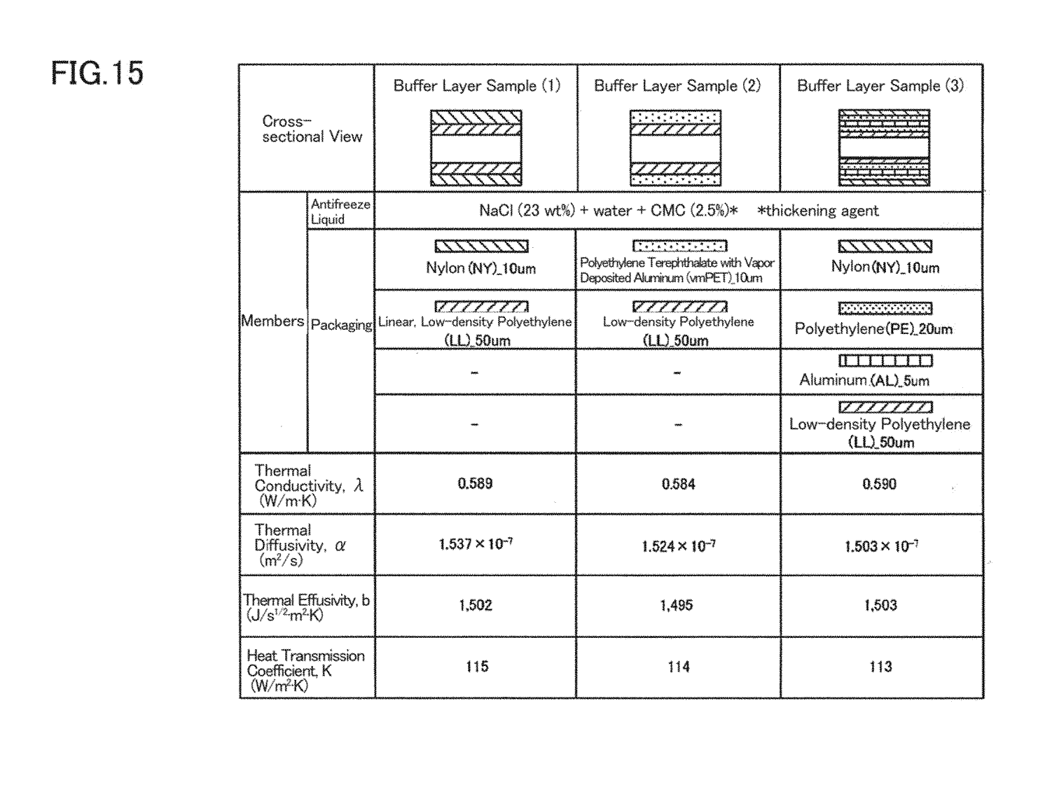

FIG. 13 is an illustration of the condition of a cooling pack prepared using a supporter and worn around the arm. FIG. 14 is an example questionnaire in which the level of comfort/discomfort is marked on a scale every time a unit time has elapsed. A cooling pack was secured around the arm using a supporter as shown in FIG. 13, and the level of comfort/discomfort was evaluated every time a unit time had elapsed as shown in FIG. 14. More specifically, the three samples with different buffer layer structures shown in FIG. 15 (buffer layer samples (1) to (3)) were prepared, and the comfort/discomfort levels of the three samples worn were evaluated using the questionnaire. The scale was from 0 for uncomfortable to 10 for comfortable, and respondents were asked to mark the level of comfort/discomfort they felt on the scale. They were also asked to do the same every time a unit time had elapsed (every 1 minute in this evaluation) to keep track of how their perceived level of comfort/discomfort changed over time. As indicated in FIG. 15, the thermophysical property values employed in the present invention may have certain ranges. Specifically, the thermal conductivity may range from 0.584 to 0.590, the thermal diffusivity from 1.503×10⁻⁷ to 1.537×10⁻⁷, the thermal effusivity from 1,495 to 1,503, and the heat transmission coefficient from 113 to 115.

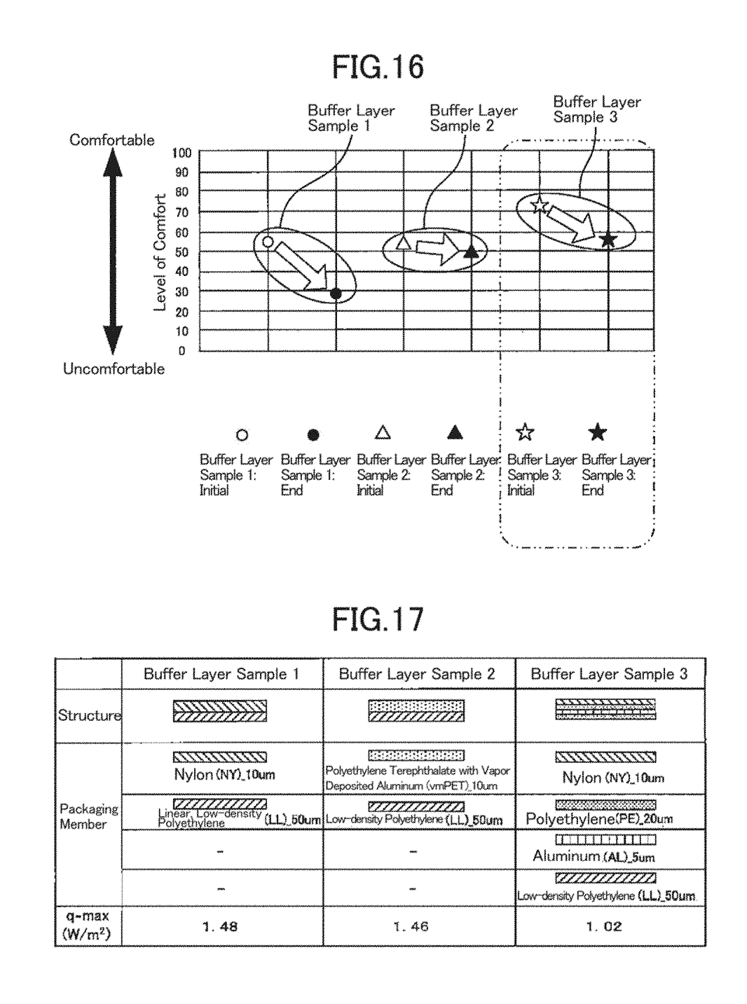

FIG. 16 is a diagram representing results of sensory evaluations. Referring to FIG. 16, “Sample 1: Initial” indicates the comfort/discomfort level felt by the subject immediately after the subject put on a cooling pack that contained Buffer Layer Sample 1, and “Sample 1: End” indicates the comfort/discomfort level 10 minutes after the subject put on the cooling pack. The same description applies to samples 2 and 3. FIG. 16 demonstrates that Buffer Layer Sample 3, or the cooling pack including the buffer layer that had the thermophysical property values given in Example 1 and the cold storage layer that had a phase transition temperature of 12° C., was the most comfortable to wear.

FIG. 17 is a table of the structures of buffer layer samples and the compositions and q-max values of packaging members. Each packaging member was placed on a constant temperature plate, and contact cold feeling (q-max) was measured using sensors. In this context, temperature change is given by the equation: ΔT=Tₛₑₙₛₒᵣ−T_{constant-temperature-plate}=20° C. Resultant measurements are shown in the bottom row of FIG. 17. Buffer Layer Sample 3 exhibited the lowest values, which indicates that it removed less heat than the other two samples. To put it differently, Buffer Layer Sample 3 is less likely to give cold sensation when brought into contact with the human body.

### EXAMPLE 2

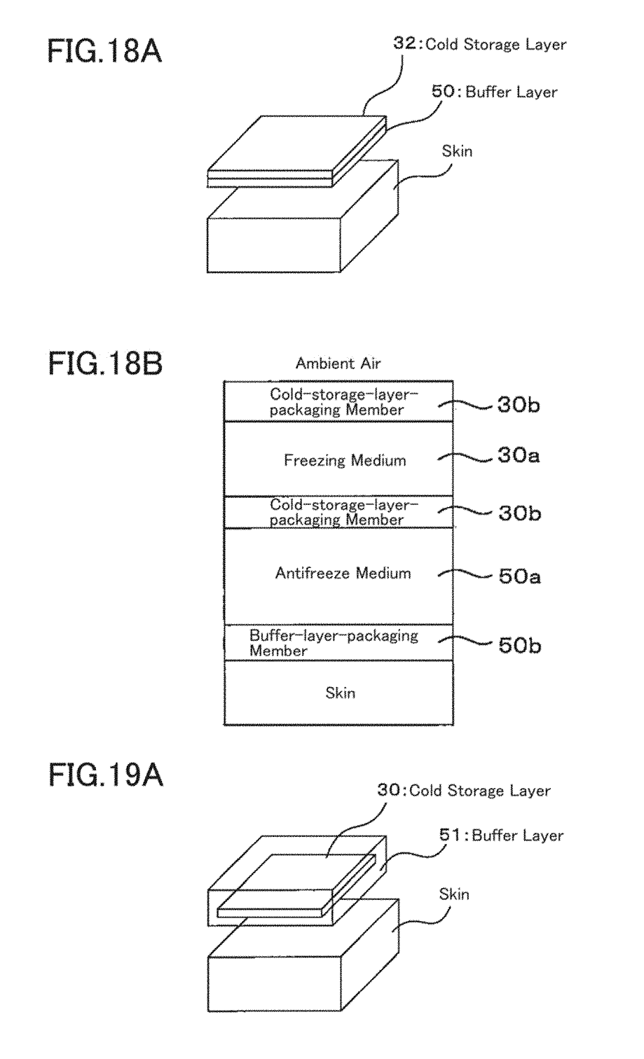

FIGS. 18A and 18B are schematic diagrams of a structure of a cooling pack in accordance with Example 2. In Example 2, the antifreeze medium 50a is encased between the cold-storage-layer-packaging member 30b and the buffer-layer-packaging member 50b. This structure enables efficient heat transfer between the freezing medium and the antifreeze medium and also improves the integral construction of the cooling pack.

### EXAMPLE 3

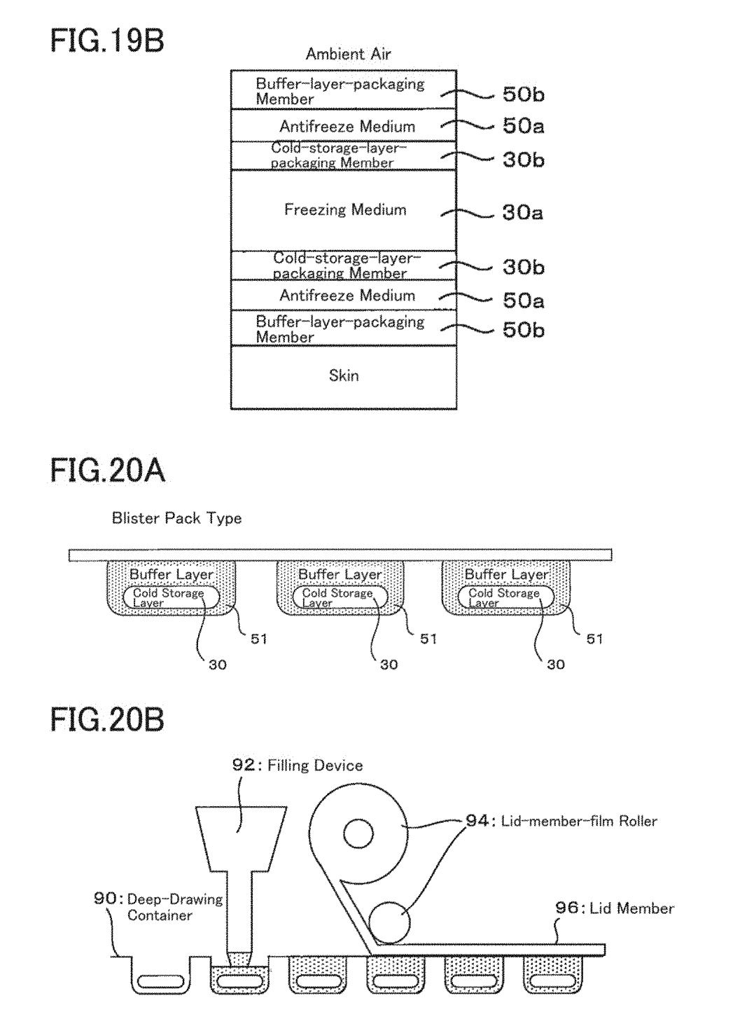

FIGS. 19A and 19B are schematic diagrams of a structure of a cooling pack in accordance with Example 3. In Example 3, the freezing medium 30a and the cold-storage-layer-packaging member 30b encasing the freezing medium 30a are encased in a buffer layer 51. This structure prohibits the cold storage layer 30 from coming into contact with ambient air on the side opposite from the skin, thereby preventing the cold storage layer 30 from collecting heat from ambient air. That in turn prolongs the cooling time achieved by the cold storage layer 30.

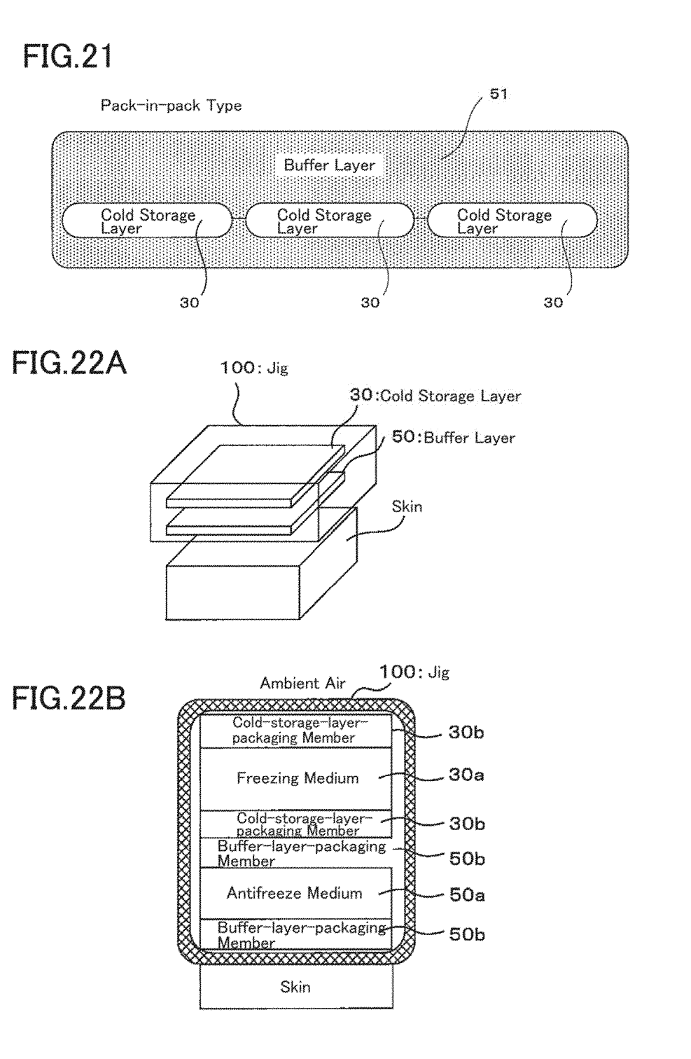

FIGS. 20A and 20B are diagrams of a process of manufacturing the cooling pack in accordance with Example 3. The cooling pack in accordance with Example 3 may be fabricated in the form of blister pack. The cold storage layer 30 is encased in the buffer layer 51 as shown in FIG. 20A. Referring to FIG. 20B, the blister pack is prepared by placing the cold storage layer 30 inside a deep-drawing container 90, filling the deep-drawing container 90 with an antifreeze medium from a filling device 92, and feeding a lid member 96 from a lid-member-film roller. FIG. 21 is a diagram of the cooling pack in accordance with Example 3 prepared as a pack-in-pack model. A plurality of cold storage layers 30 is coupled in an in-plane direction and encased in the buffer layer 51 as shown in FIG. 21. These structures of the cooling pack prepared as a blister pack and a pack-in-pack as described here also prohibit the cold storage layer 30 from coming into contact with ambient air on the side opposite from the skin, thereby preventing the cold storage layer 30 from collecting heat from ambient air. That in turn prolongs the cooling time achieved by the cold storage layer 30.

### EXAMPLE 4

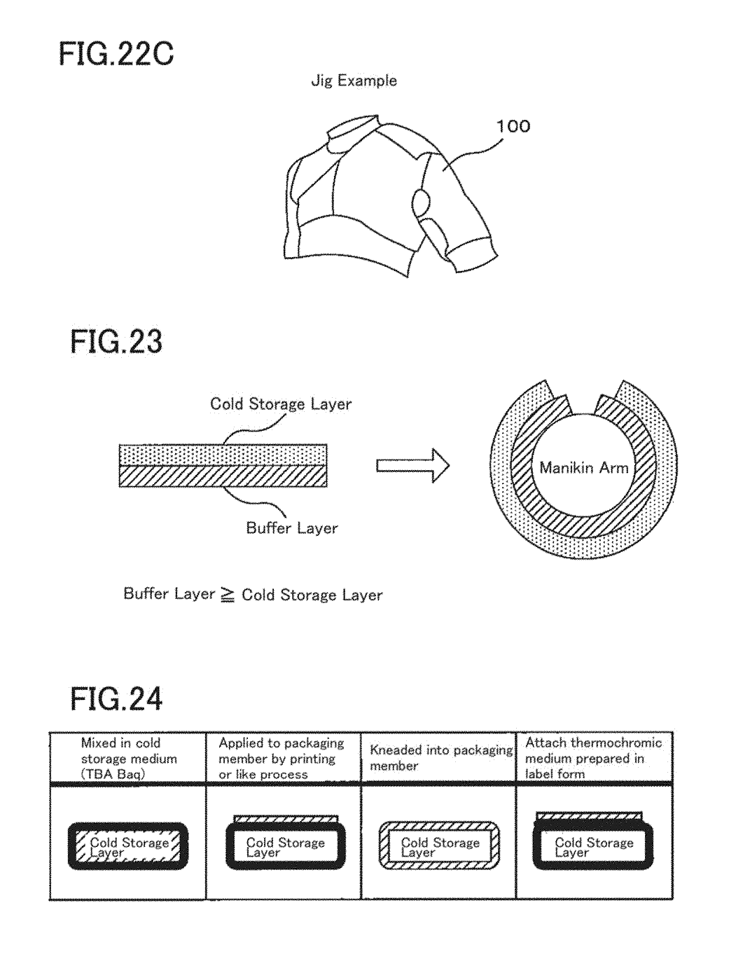

FIGS. 22A and 22B are schematic diagrams of a structure of a cooling pack and a therapeutic tool in accordance with Example 4. In Example 4, the cooling pack in any one of Examples 1 to 3 is arranged such that the cooling pack can be attached to a part of a human body in a fixed manner using a jig 100. The jig 100 may be, for example, a supporter or a towel. FIG. 22C is an illustration of a usage example. Configuring a therapeutic tool from a cooling pack and the jig 100 in this manner enables effective cooling therapy.

### Thermal Insulation Layer

The cooling pack in any one of Examples 1 to 3 may include a thermal insulation layer on the cold storage layer on the side opposite from the buffer layer. This structure prohibits the cold storage layer from coming into contact with ambient air on the side opposite from the skin, thereby preventing the cold storage layer from collecting heat from ambient air. That in turn prolongs the cooling time achieved by the cold storage layer.

### Prevention of Non-Uniform Temperature Distribution.

The inventors of the present Invention have also found that the in-plane lengths of the buffer layer and the cold storage layer can in some cases influence cooling effects. More specifically, if the cold storage layer has a larger in-plane length than that of the buffer layer, the cold storage layer may become excessively long when the cooling pack is attached to the human body. The cold storage layer with an excess length is warmed up when brought into contact with the skin, causing a non-uniform temperature distribution. Since the cold storage layer has a lower temperature than the buffer layer, the cold storage layer may, if brought into direct contact with the skin, cause the skin to be cooled down to the noxious cold stimulus region shown in FIG. 12, possibly falling short of achieving the object of the present invention. The cooling pack is therefore designed such that the buffer layer has an in-plane length that is larger than or equal to that of the cold storage layer when the cold storage layer and the buffer layer are stacked as shown in FIG. 23. This design enables the buffer layer to be unfailingly longer than the cold storage layer when the cooling pack is attached to the human body.

### Thermochromic Medium

The cooling pack in accordance with the present embodiment may include a thermochromic medium, which is also advantageous. A thermochromic medium is a substance that changes color with temperature. Thermochromic media come in various temperature ranges, colors, and forms and are commercially available as listed in the following table.

TABLE 2

| | |
|---|---|
| Capsule Slurry | May be mixed with a fixing agent in dispersed water to prepare an |
| | aqueous paint or ink. This paint or ink may be absorbed by blank T-shirts |
| | so that the clothes can change color with body temperature. |
| Capsule Powder | Fine (several micrometers) particulate powder. May be mixed with an |
| | oil-based binder (fixing agent) to prepare an ink for printing on, for |
| | example, film, glass, and metal. |
| Master Batch | Sold as pellets of polypropylene or a like plastic. The resin may be |
| | increased in volume by 5 to 10 folds and injection-molded to make, for |
| | example, bath toys, color-pattern-changing mugs, temperature-indicating |
| | containers for frozen food, and cold drink containers indicating whether |
| | the drink is ready to drink. Injection Mold temperature is 200° C. or lower. |
| Aqueous Screen | Suitable for printing on T-shirts or like clothes. Best to use an 80- to |
| Ink | 100-mesh screen. Best to subject to heat drying at 110° C. to 120° C. for |
| | approximately 3 minutes or at 150° C. to 160° C. for approximately 1 minute |
| | after printing. |
| Oil-based Screen | Suitable for printing on, for example, plastic films. Best to subject to heat |
| Ink | drying at 40° C. to 60° C. for approximately 3 minutes after printing. |
| Aqueous Ink | Suitable for printing on T-shirts or like clothes. Best to use an 80- to |
| | 100-mesh screen. Best to subject to heat drying at 110° C. to 120° C. for |
| | approximately 3 minutes or at 150° C. to 160° C. for approximately 1 minute |
| | after printing. |
| Oil-based Ink | Suitable for printing on, for example, plastic films. Best to subject to heat |
| | drying at 40° C. to 60° C. for approximately 3 minutes after printing. |

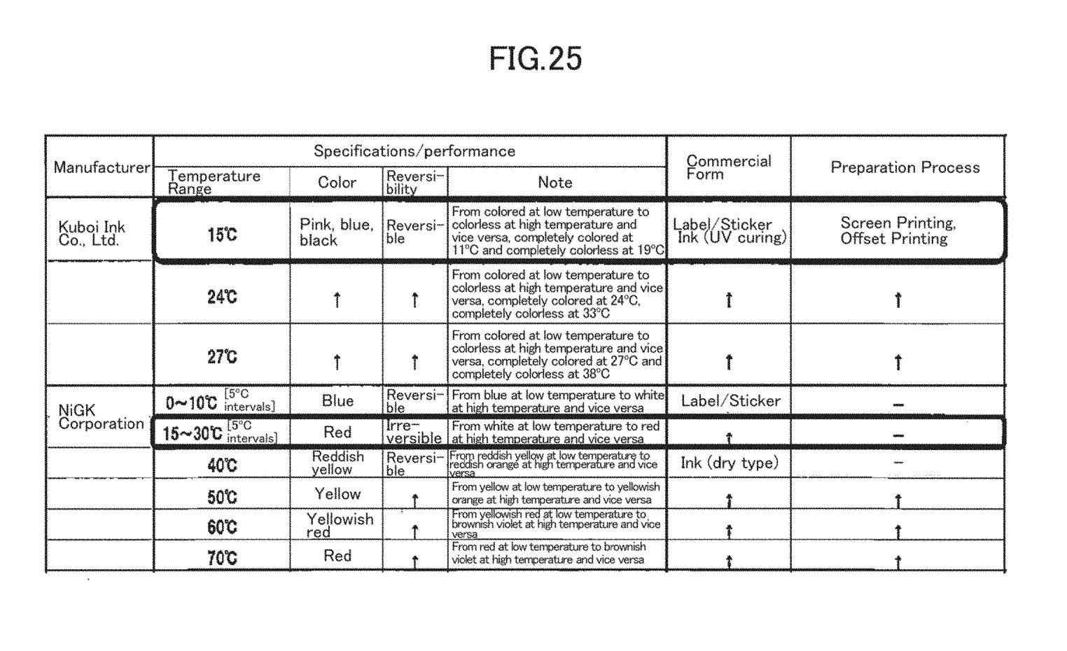

The cooling pack in accordance with the present embodiment is preferably a thermochromic medium that colors at 10° C., takes on a neutral color at 15° C., and becomes colorless at 20° C. FIG. 24 is a diagram showing a thermochromic medium being applied to cold storage layers. From left to right are shown a thermochromic medium mixed in a cold storage medium, a thermochromic medium applied by printing or a like process onto a cold-storage-layer-packaging member, a thermochromic medium kneaded into a cold-storage-layer-packaging member, and a thermochromic medium prepared as a label and attached onto a cold-storage-layer-packaging member. Using such a thermochromic medium, the cold storage layer has a color at optimal temperature (12° C.), and becomes colorless when the temperature rises and exceeds 20° C. after cooling is over. This arrangement enables visual recognition of cooling pack temperature. FIG. 25 lists preferred thermochromic media for the cooling pack in accordance with the present embodiment. The two thermochromic media encircled with thick lines in FIG. 25 are optimal examples.

### EXAMPLE 5

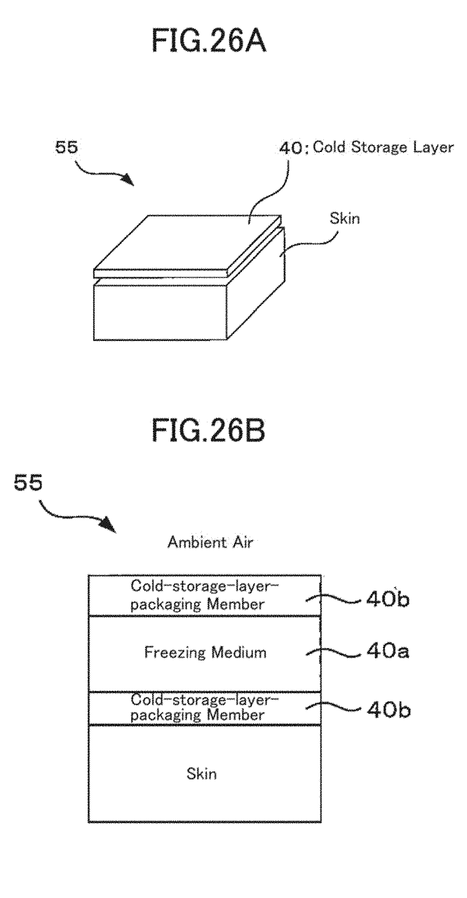

FIGS. 26A and 26B are schematic diagrams of a structure of a cooling pack in accordance with Example 5. A cooling pack 55 in accordance with Example 5 includes only a cold storage layer 40 and no buffer layer. Specifically, a cold-storage-layer-packaging member 40b encases a freezing medium 40a as shown in FIG. 26B.

### Skin Temperature Measurement Experiment

A pseudo-skin temperature measurement experiment was conducted on the cooling pack 55 in accordance with Example 5 using a thermal manikin as shown earlier in FIGS. 10A and 10B. The thermal manikin 80 has a function of artificially reproducing the generation of heat by a human body at ambient temperature. The cooling pack 55 was manufactured in accordance with Example 5 in this example. Specifically, the freezing medium 30a was 40 wt % TBAB, and the cold-storage-layer-packaging member 30b was made of nylon polyethylene with a thickness of 60 μm. A cold storage layer 32 weighed 350 grams. This cooling pack 55 was attached to the thermal manikin 80, and changes in the temperature of the artificial skin surface were measured by the thermographic camera 82. In this experiment, a surface temperature distribution was obtained from the surface of the thermal manikin 80 (=skin surface) immediately after the cooling pack 55 was removed.

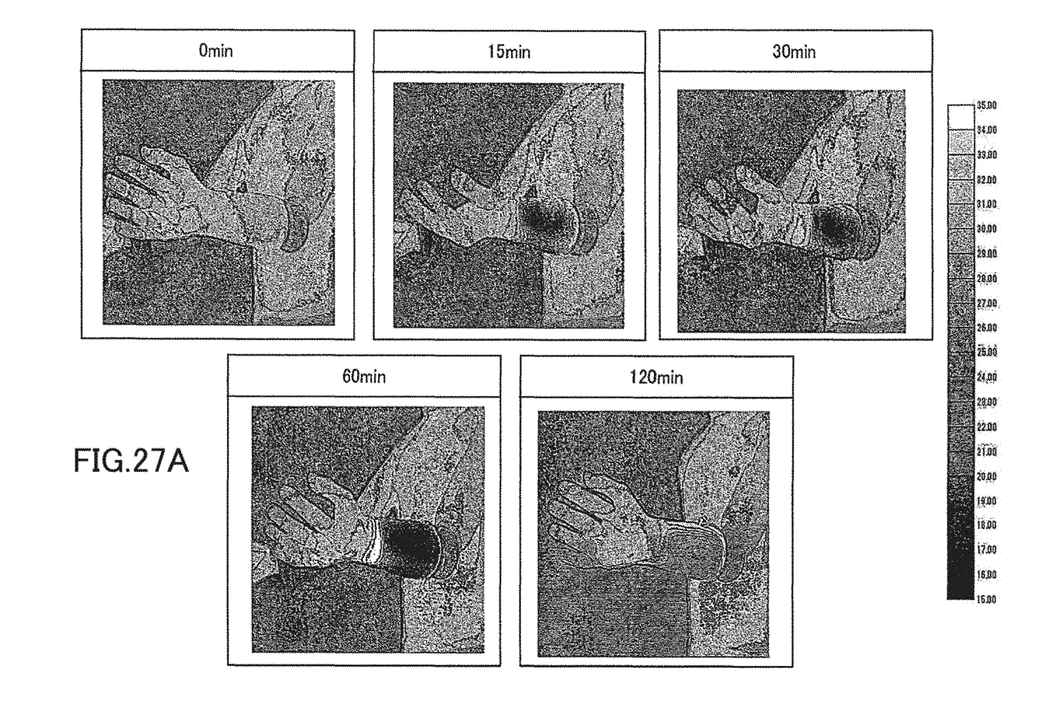

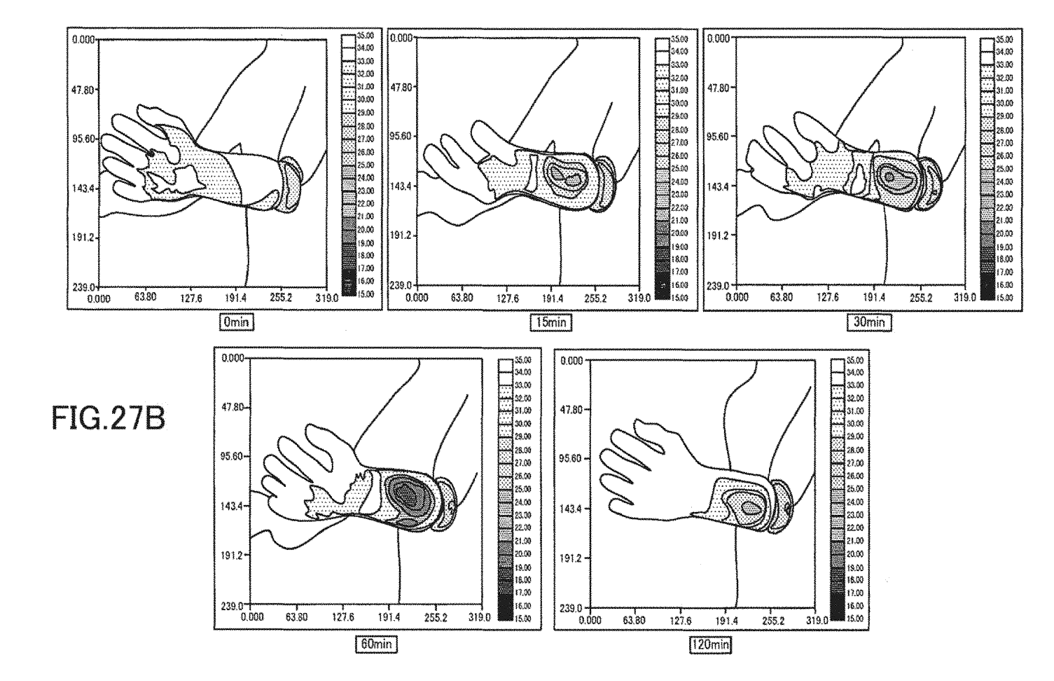

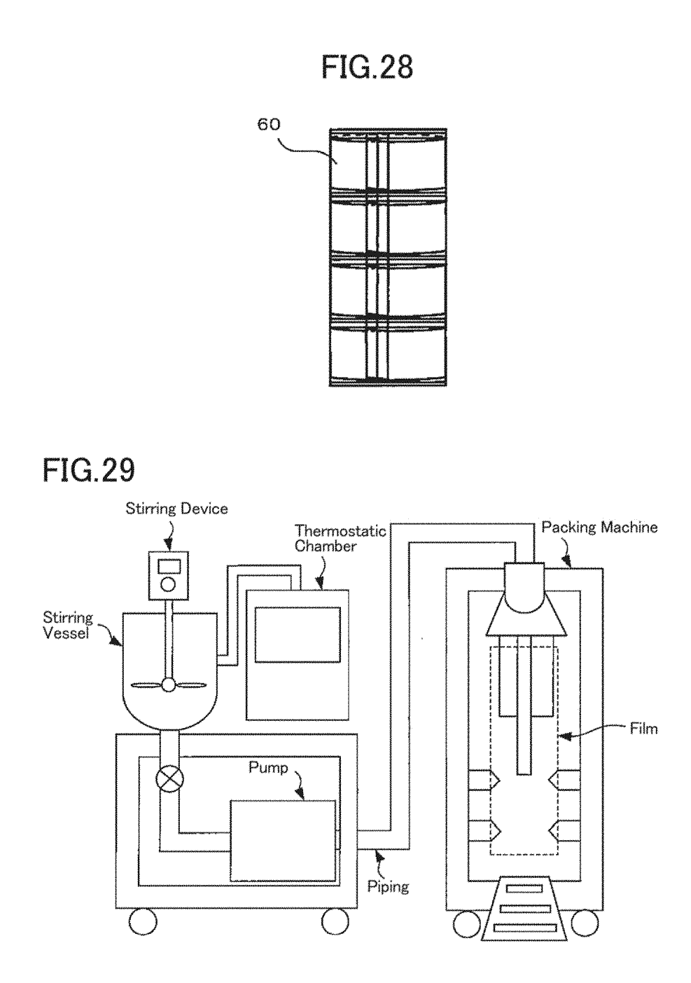

FIG. 27A is a set of diagrams (contour figures) showing results (measurements) of a pseudo-skin temperature measurement experiment using a thermal manikin. FIG. 27B is a line map version of FIG. 27A. FIGS. 27A and 28B selectively show measurements taken 0, 15, 30, 60, and 120 minutes after the measurement was started. FIGS. 27A and 27B demonstrate that even without a buffer layer, the skin temperature does not go into the noxious cold stimulus region (region at or below 17° C.) and is maintained at around 20° C., which is suitable to the human body, for 120 minutes. The cooling pack in accordance with Example 5 weighed 350 grams, which is much lighter than a cooling pack including both a cold storage layer and a buffer layer that weighs approximately 500 grams. The lightweight cooling pack 55 reduces the load when the cooling pack 55 is attached and while the cooling pack 55 is being worn. The lightweight cooling pack 55 also contributes to cost reduction.

### EXAMPLE 6

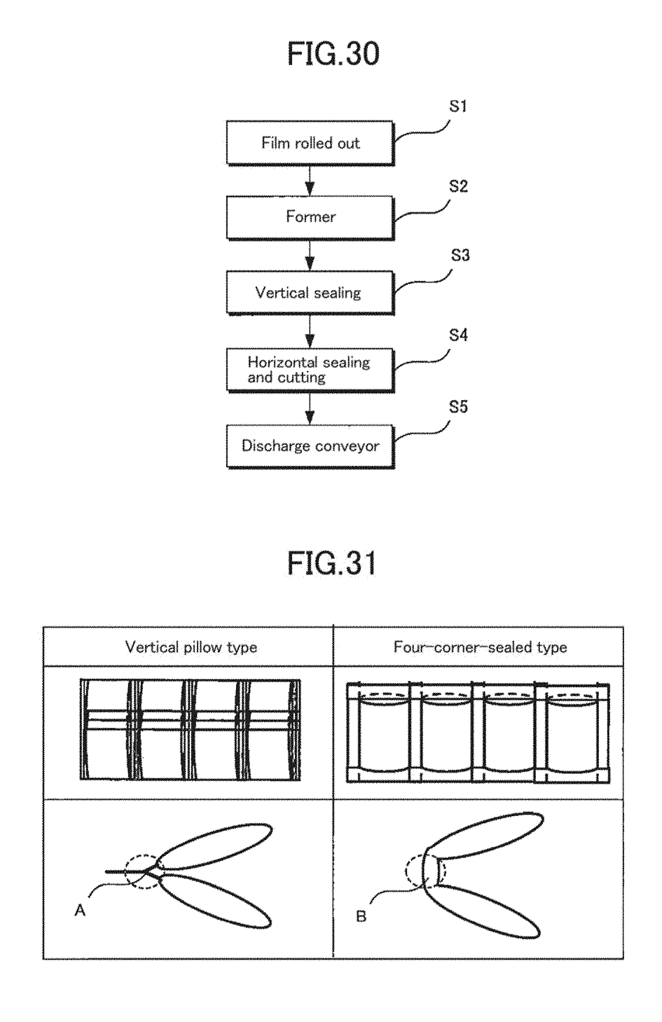

FIG. 28 is a schematic diagram of a structure of a cooling pack in accordance with Example 6. A cooling pack 60 in accordance with Example 6 includes only a cold storage layer similarly to the cooling pack in accordance with Example 5 and is manufactured in a vertical form-fill seal machine. FIG. 29 is a schematic illustration of an automatic packing machine for manufacturing a cooling pack in accordance with Example 6. FIG. 30 Is a diagram representing a process of manufacturing the cooling pack 60 in a vertical form-fill seal machine. The vertical form-fill seal machine may be, for example, one of those listed in Food Packing Technology Handbook (“Shokuhin Housou Gijutsu Binran”). Referring to FIG. 30, a film is first rolled out (step S1), and both ends (sealing surfaces) of the film is joined together in a former (step S2). The film is then subjected to vertical sealing to form a tube (step S3). The film is pulled down a unit at a time and subjected to horizontal sealing by using a sucker or a film-pull and horizontal sealer. The film is then pulled down, and a weighed-out amount of freezing medium is simultaneously dropped. The resultant object is simultaneously sealed and cut (step S4) and finally discharged on a discharge conveyor (step S5).

The manufacture of a cooling pack described above involves less sealing than hand sealing, which reduces the possibility of leakage. Since the cooling pack 60 in accordance with Example 6 is manufactured in a vertical form-fill seal machine, the cooling pack 60 is folded into two by pinching vertical sealed sections and frozen in the folded-up condition. This method allows the manufacture of a cooling pack that can be bent in two axial directions.

FIG. 31 is a diagram showing a comparison of a cooling pack of a vertical pillow type and a cooling pack of a four-corner-sealed type, the former being in accordance with Example 6. FIG. 31 shows a cooling pack of a vertical pillow type on the left side and a cooling pack of a four-corner-sealed type on the right side. The cooling pack of a four-corner-sealed type is sealed on three sides. Both types are folded into two and frozen in that condition. The cooling pack of a vertical pillow type allows for the clear and easy formation of vertical folding lines by pinching vertically sealed segments and folding the cooling pack into two. On the other hand, if a cooling pack of a four-corner-sealed type is folded into two, it is difficult to form a clear folding line.

A comparison of the cross-sections of the folding lines of the two types shows that a cooling pack of a vertical pillow type has no freezing medium remaining along the folding lines when folded up as indicated by “A” in FIG. 31 and therefore can be easily expanded even after being frozen. On the other hand, a cooling pack of a four-corner-sealed type has a freezing medium remaining along the folding lines when folded up as indicated by “B” in FIG. 31. If the cooling pack is frozen in this condition, the cooling pack cannot be easily expanded and fails to provide sufficient convenience as a cooling pack.

### Skin Temperature Measurement Experiment

A pseudo-skin temperature measurement experiment was conducted on the cooling pack 60 in accordance with Example 6 using a thermal manikin as shown earlier in FIGS. 10A and 10B. Tie thermal manikin 80 has a function of artificially reproducing the generation of heat by a human body at ambient temperature. As described earlier, the cooling pack in accordance with Example 6 is of a vertical pillow type. The freezing medium and cold-storage-layer-packaging member were the same as those in Example 5.

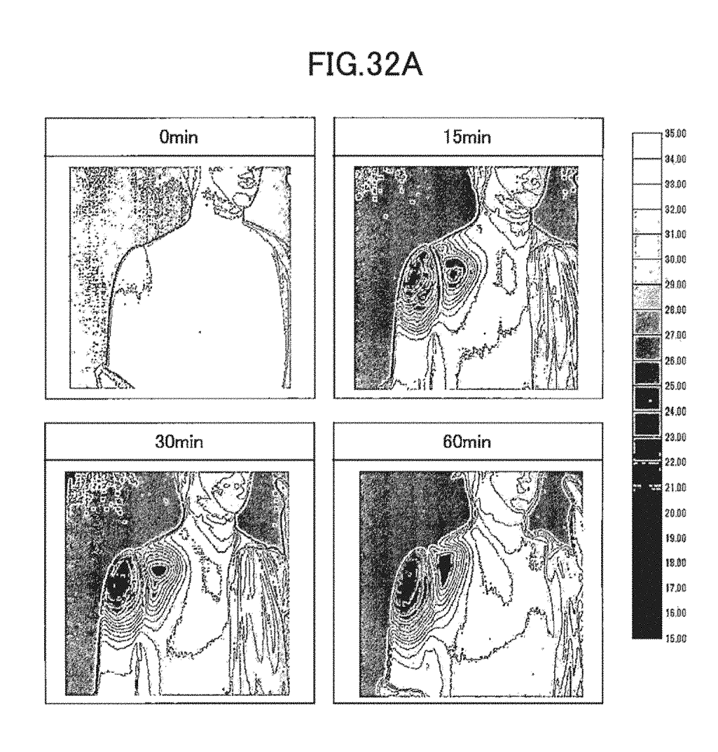

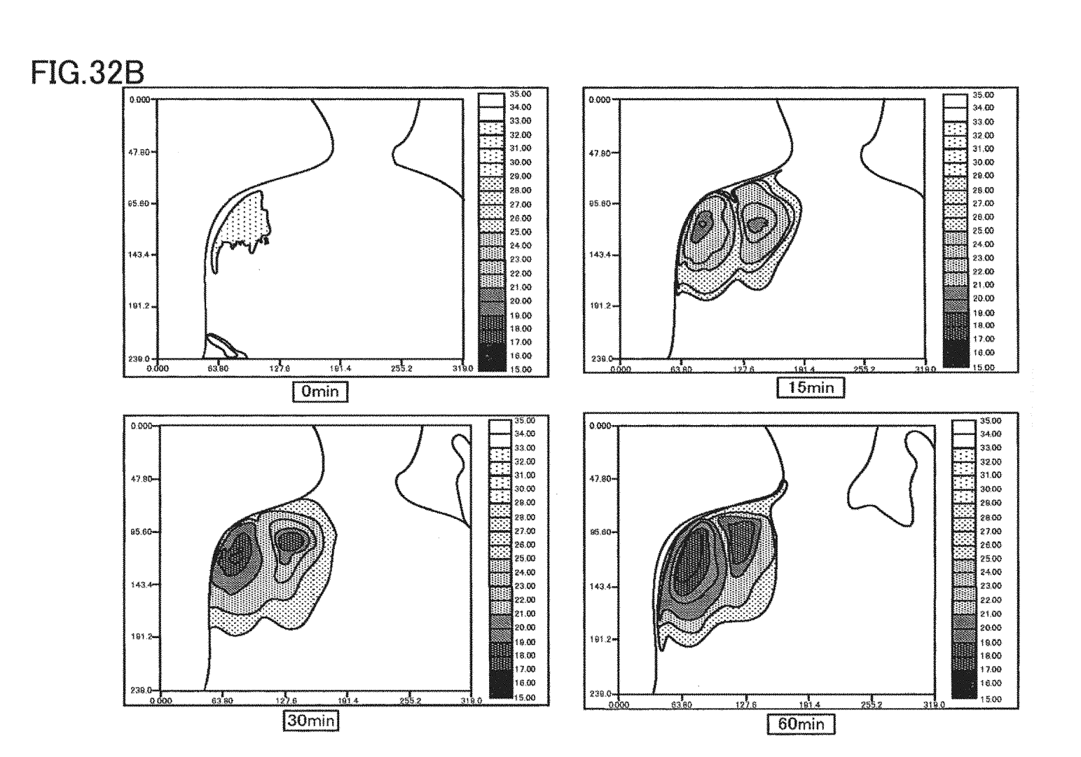

FIG. 32A is a set of diagrams (coutour figures) showing results (measurements) of a pseudo-skin temperature measurement experiment using a thermal manikin. FIG. 32B is a line map version of FIG. 32A. FIGS. 32A and 32B selectively show measurements taken 0, 15, 30, and 60 minutes after the measurement was started. Cooling packs that can be bent only in one axial direction are wound, for example, around the foot or arm, whereas cooling packs that can be bent in two axial directions as in Example 6 can be used to enwrap the site to be kept cool. FIG. 32 shows results of experimental enwrapped cooling of a shoulder of a thermal manikin. FIG. 32 demonstrates that the skin temperature does not go into the noxious cold stimulus region (region at or below 17° C.) and is maintained at around 20° C., which is suitable to the human body, for 60 minutes.

### Palm Cooling

Palm cooling has been conventionally known as a method of cooling for the prevention of heatstroke. This method cools the palm, which in turn cools blood. The cooled blood returns to the heart and circulates throughout the body. Deep body temperature is lowered by this cycle. A palm cooling device has been suggested that circulates ice water in order to adjust the contact temperature of the palm to 12° C. to 15° C.

If the palm cooling temperature is lower than 12° C. to 15° C., blood vessels contract, which makes it difficult to lower deep body temperature. On the other hand, if the palm cooling temperature is higher than this temperature range, no cooling can be achieved. For these reasons, the freezing medium in accordance with the present embodiment is TBAB, which has a melting point of 12° C. and allows for specification of a suitable cooling temperature. In addition, the cooling pack in accordance with the present embodiment needs no circulation mechanism or like accessories, which allows for reduction in weight, size, and cost and maintenance of a desired temperature range over an extended period of time.

### EXAMPLE 7

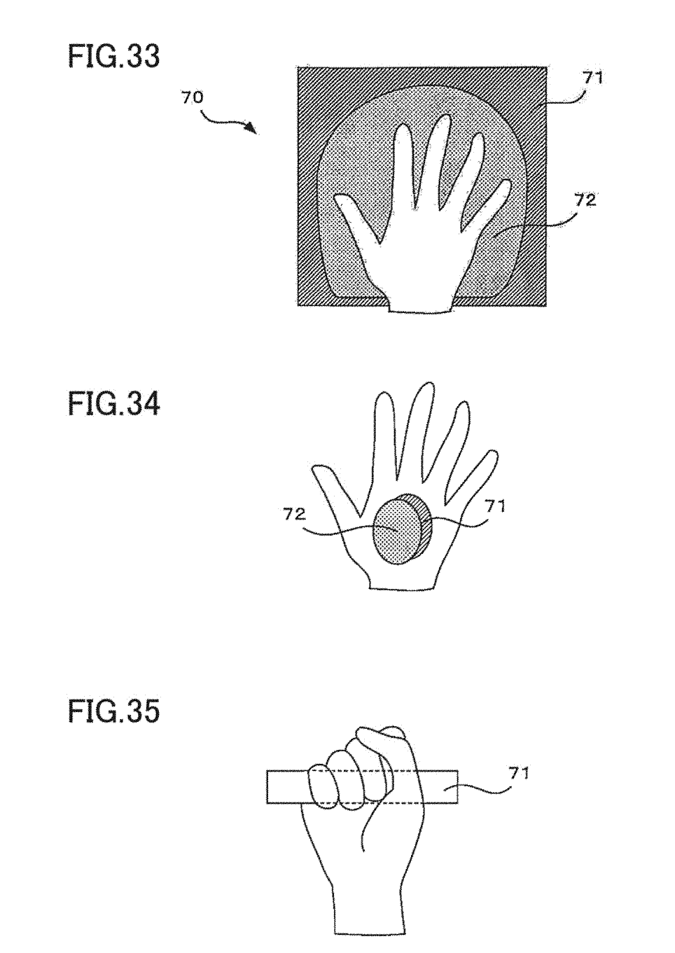

FIG. 33 is an illustration of a cooling pack for palm cooling in accordance with Example 7. This cooling pack 70 has such a size and shape as to envelop a human hand. The cooling pack 70 Includes an outer cold storage layer 71 and an Inner buffer layer 72. This provision of the buffer layer 72 allows cold air to flow between digits. More specifically, the cooling pack 70 includes: a mitten section made of a flexible material and shaped like a bag to envelop a human hand; and the buffer layer 72 made of an antifreeze medium that is flexible at the phase transition temperature of a freezing medium. The buffer layer 72 is provided in a thickness direction of the cooling pack 70 (mitten section) and constitutes an antifreeze-medium-holding section that holds the antifreeze medium. The buffer layer 72 is brought Into contact with human skin for heat transfer between the human body and the cold storage layer.

### EXAMPLE 8

FIG. 34 is an illustration of a cooling pack for palm cooling in accordance with Example 8. In Example 8, the cooling pack includes a cold storage layer 71 and a buffer layer 72 and cools only the palm. The cooling pack does not enwrap the digits. This structure therefore allows the user to move his/her digits during cooling. The buffer layer 72 may be omitted in Example 8.

### EXAMPLE 9

FIG. 35 is an illustration of a cooling pack for palm cooling in accordance with Example 9. In Example 9, the cooling pack includes only a cold storage layer 71 shaped like a stick and cools only the palm. The user can have his/her palm cooled by holding the stick-like cold storage layer 71 in his/her hand. The buffer layer 72 may be omitted in Example 8.

### EXAMPLE 10

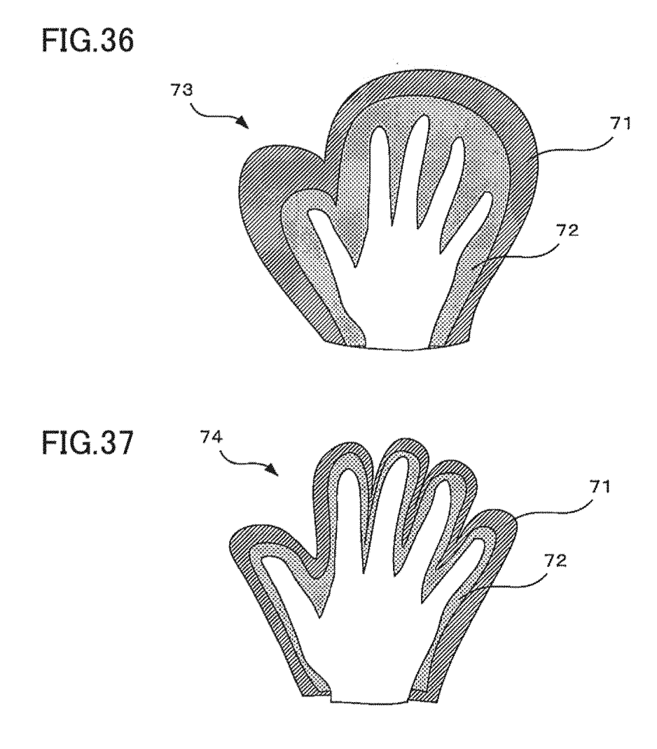

FIG. 36 is an illustration of a cooling pack for palm cooling in accordance with Example 10. In Example 10, the cooling pack is shaped like a mitten. This cooling pack 73 has such a size and shape as to envelop a human hand. The cooling pack 73 includes an outer cold storage layer 71 and an inner buffer layer 72. This provision of the buffer layer 72 allows cold air to flow between digits.

### EXAMPLE 11

FIG. 37 is an illustration of a cooling pack for palm cooling in accordance with Example 11. In Example 11, the cooling pack is shaped like a glove. This cooling pack 74 has such a size and shape as to envelop a human hand. The cooling pack 74 is further designed to cool each digit separately. The cooling pack 74 includes an outer cold storage layer 71 and an inner buffer layer 72. This provision of the buffer layer 72 allows cold air to flow between digits.

### EXAMPLE 12

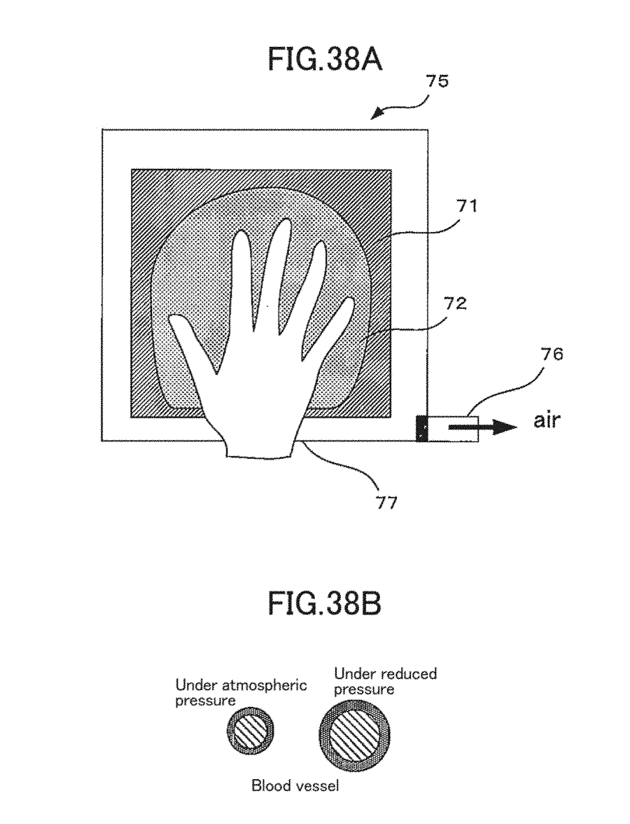

FIG. 38A is an illustration of a cooling pack for palm cooling in accordance with Example 12. A cooling pack 75 in accordance with Example 12 has such a size and shape as to envelop a human hand. The cooling pack 75 includes an outer cold storage layer 71 and an inner buffer layer 72. The cooling pack 75 is further designed to have a function of blocking an incoming flow of ambient air at an open/close section 77 for reduced pressure. Referring to FIG. 38A, the open/close section 77 is closed, and air is sucked through a pipe 76, to reduce the internal pressure of the cooling pack 75 to a level below atmospheric pressure. FIG. 38B is an illustration of a cross-section model of a blood vessel. The blood vessel expands under reduced pressure when compared with under atmospheric pressure. The interior of the cooling pack is depressurized to expand blood vessels. That in turn increases the blood flow, hence the amount of cooled blood, which leads to efficient lowering of deep body temperature.

### EXAMPLE 13

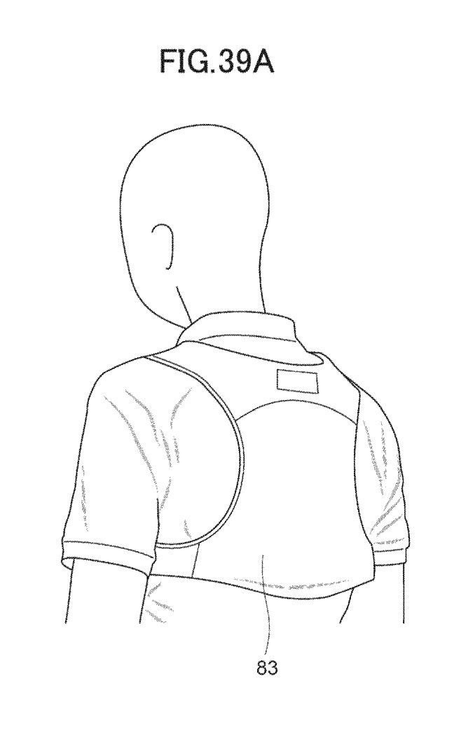

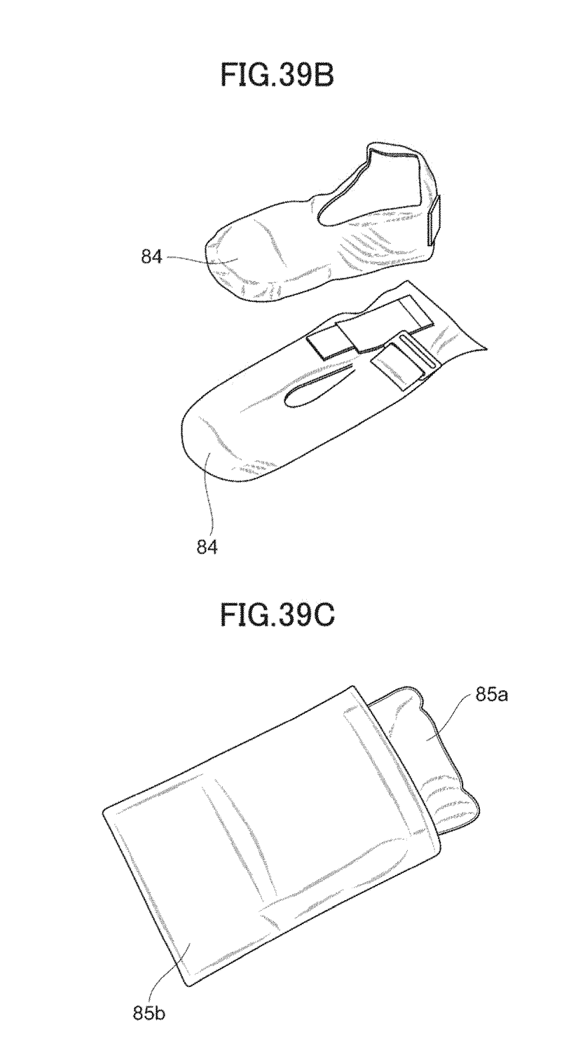

FIG. 39A is an illustration of a cooling pack in accordance with Example 13. In Example 13, a cooling pack 83 is shaped like a rucksack so that the user can carry it on his/her back. This carry-on-the-back design of the cooling pack 83 allows the user to move easily and achieves large area cooling. Hence, when the user engages in an activity under the hot sun, the cooling pack 83 effectively cools his/her body, thereby preventing body temperature rises.

### EXAMPLE 14

FIG. 39B is an illustration of a cooling pack in accordance with Example 14. In Example 14, a cooling pack 84 is shaped like a slipper so that the user can wear it on his/her foot. This slip-on design renders the cooling pack 84 less likely to come off when the user moves around in the cooling pack 84 and also achieves the cooling of the entire foot. The cooling pack 84 can hence provide continuous and stable cooling, for example, for a bruised foot.

### EXAMPLE 15

FIG. 39C is an illustration of a cooling pack in accordance with Example 15. In Example 15, a cooling pack 85a is shaped like a pillow. The user can put the pillow-shaped cooling pack 85a in a cover 85b for use. This use of the cooling pack 85a as a pillow enables the user to cool his/her head, for example, during sleep. In other configurations, the cooling pack in accordance with the present embodiment may be disposed inside a helmet, a cap, or a like head gear to wear on his/her head to cool his/her head. The cooling pack may be attached to the head in a fixed manner, for example, by disposing the cooling pack in accordance with the present embodiment inside a helmet and fastening a chin strap. This configuration enables the cooling of the head when the user is engaging in an activity in a high temperature environment. The examples given so far have described cooling of the hand, the foot, and other parts of the body. Alternatively, one can also concentrate on cooling a site where there are many arteriovenous connections. An arteriovenous connection is a direct connection of an artery and a vein without intervening capillary blood vessels. Deep body temperature can be efficiently cooled by cooling the sites where there are many arteriovenous connections.

### EXAMPLE 16

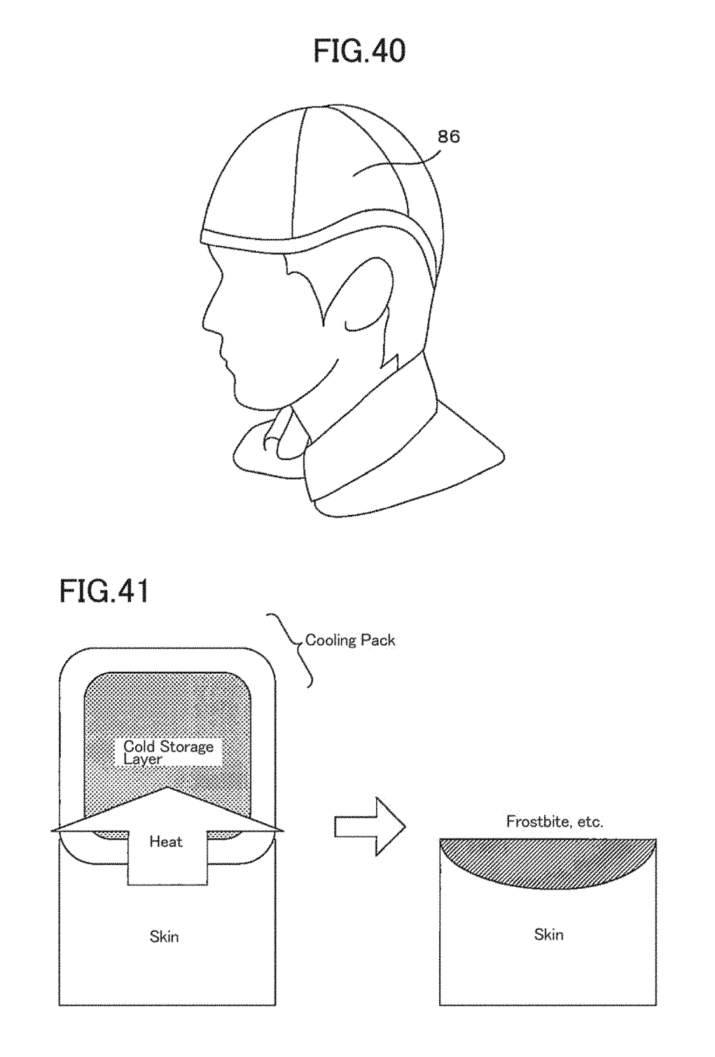

FIG. 40 is an illustration of a cooling pack in accordance with Example 16. In Example 16, a cooling pack 86 is shaped like a cap. This put-on design of the cooling pack 86 achieves the cooling of the entire head.

(A) The present invention may have the following aspects. The present invention, in one aspect thereof, is directed to a cooling pack that cools a human body, the cooling pack including: a freezing medium having a phase transition temperature specified in a range of temperature, the range being suitable to maintain the human body at low temperature; and a first container section containing the freezing medium therein, wherein the freezing medium and the first container section constitute a cold storage layer.

This cooling pack, as described here, includes: a freezing medium having a phase transition temperature specified in a range of temperature, the range being suitable to maintain the human body at low temperature; and a first container section containing the freezing medium therein, wherein the freezing medium and the first container section constitute a cold storage layer. Therefore, the cooling pack can both maintain the human body at a suitable, low temperature and ensure a sufficient usage time.

(B) The cooling pack in another aspect of the present invention farther includes: an antifreeze medium flexible at the phase transition temperature of the freezing medium; and a second container section including a flexible material and containing the antifreeze medium therein, wherein: the antifreeze medium and the second container section constitute a buffer layer having a prescribed thermal conductivity, a prescribed thermal diffusivity, a prescribed thermal effusivity, and a prescribed heat transmission coefficient; and the buffer layer transfers heat between the human body and the cold storage layer when brought into contact with skin of the human body.

This cooling pack, as described here, further includes: an antifreeze medium flexible at the phase transition temperature of the freezing medium; and a second container section including a flexible material and containing the antifreeze medium therein, wherein: the antifreeze medium and the second container section constitute a buffer layer having a prescribed thermal conductivity, a prescribed thermal diffusivity, a prescribed thermal effusivity, and a prescribed heat transmission coefficient; and the buffer layer transfers heat between the human body and the cold storage layer when brought into contact with skin of the human body. Therefore, the cooling pack can both maintain the human body at a suitable, low temperature and ensure a sufficient usage time.

(C) The cooling pack in another aspect of the present invention maintains a site in contact with the skin of the human body at or above 17° C.

This cooling pack, as described here, maintains a site in contact with the skin of the human body at or above 17° C. Therefore, the cooling pack is capable of cooling over an extended period of time without activating the TRPA1 channel of the human body.

(D) The cooling pack in another aspect of the present invention is such that regarding the buffer layer, the thermal conductivity is from 0.584 to 0.590, the thermal diffusivity is from 1.503×10⁻⁷ to 1.537×10⁻⁷, the thermal effusivity is from 1,495 to 1,503, and the heat transmission coefficient is from 113 to 115.

This cooling pack, as described here, includes the buffer layer having these prescribed thermophysical property values. Therefore, the cooling pack is capable of maintaining the human body at a suitable, low temperature.

(E) The cooling pack in another aspect of the present invention is such that the cold storage layer includes: a plurality of first container sections; and an articulation mechanism configured to couple the plurality of first container sections.

This cooling pack, as described here, is such that the cold storage layer includes: a plurality of first container sections; and an articulation mechanism configured to couple the plurality of first container sections. Therefore, the cooling pack fits the human body well and is capable of uniformly cooling a large area.

(F) The cooling pack in another aspect of the present invention is such that the antifreeze medium is held by a part of the first container section and a part of the second container section.

This cooling pack, as described here, is such that the antifreeze medium is held by a part of the first container section and a part of the second container section. Therefore, the cooling pack both increases the efficiency of heat transfer between the freezing medium and the anti freeze medium and allows for integral construction of the cooling pack.

(G) The cooling pack in another aspect of the present invention is such that the cold storage layer, as well as the antifreeze medium, is encased in the second container section.

This cooling pack, as described here, is such that the cold storage layer, as well as the antifreeze medium, is encased in the second container section. Therefore, the cooling pack can prohibit the cold storage layer from coming into contact with ambient air on the side opposite from the skin, thereby preventing the cold storage layer from collecting heat from ambient air. That in turn prolongs the cooling time achieved by the cold storage layer.

(H) The cooling pack in another aspect of the present invention is such that the cold storage layer includes a thermal insulation layer on a side thereof opposite from the buffer layer.

This cooling pack, as described here, is such that the cold storage layer includes a thermal insulation layer on a side thereof opposite from the buffer layer. Therefore, the cooling pack can prohibit the cold storage layer from coming into contact with ambient air on the side opposite from the skin, thereby preventing the cold storage layer from collecting heat from ambient air. That in turn prolongs the cooling time achieved by the cold storage layer.

(I) The cooling pack in another aspect of the present invention is such that the cold storage layer includes: a plurality of first container sections arranged in a matrix on a plane; and a plurality of articulation mechanisms configured to couple the plurality of first container sections.

This cooling pack, as described here, is such that the cold storage layer includes: a plurality of first container sections arranged in a matrix on a plane; and a plurality of articulation mechanisms configured to couple the plurality of first container sections. Therefore, the cooling pack can be bent freely owing to the articulation mechanisms. Due to this structure, the cooling pack can be readily expanded even after being bent and frozen.

(J) The cooling pack in another aspect of the present invention is such that the or each articulation mechanism is configured to be freely bendable and stretchable so that the plurality of first container sections is stacked by bending the or each articulation mechanism and expanded on a plane by stretching the or each articulation mechanism.

This cooling pack, as described here, is such that the or each articulation mechanism is configured to be freely bendable and stretchable so that the plurality of first container sections is stacked by bending the or each articulation mechanism and expanded on a plane by stretching the or each articulation mechanism. Therefore, the cooling pack can be bent freely owing to the or each articulation mechanism even when frozen. Due to this structure, the cooling pack can be wrapped around the shoulder, knee, or another like site of the human body for cooling.

(K) The cooling pack in another aspect of the present invention further includes: a mitten section including a flexible material and shaped like a bag to contain a hand of the human body therein; an antifreeze medium flexible at the phase transition temperature of the freezing medium; and an antifreeze-medium-holding section provided in a thickness direction of the mitten section to hold the antifreeze medium, wherein: the mitten section, the antifreeze medium, and the antifreeze-medium-holding section constitute a buffer layer; and the buffer layer transfers heat between the human body and the cold storage layer when brought into contact with skin of the human body.

This cooling pack, as described here, further includes: a mitten section including a flexible material and shaped like a bag to contain a hand of the human body therein; an antifreeze medium flexible at the phase transition temperature of the freezing medium; and an antifreeze-medium-holding section provided in a thickness direction of the mitten section to hold the antifreeze medium, wherein: the mitten section, the antifreeze medium, and the antifreeze-medium-holding section constitute a buffer layer; and the buffer layer transfers heat between the human body and the cold storage layer when brought into contact with skin of the human body. Therefore, the cooling pack achieves palm cooling, which is an effective heatstroke-preventing method, with a simple structure.

(L) The cooling pack in another aspect of the present invention further includes: an open/close section configured to seal the mitten section; and a pipe configured to couple to a depressurizing device configured to reduce pressure in the mitten section.

This cooling pack, as described here, reduces pressure inside the cooling pack to expand blood vessels. That in turn increases the blood flow, hence the amount of cooled blood, which leads to efficient lowering of deep body temperature.

(M) The present invention, in one aspect thereof, is directed to a therapeutic tool for use in cooling therapy, the therapeutic tool including: a cooling pack according to any one of items (A) to (J); and a fixing unit configured to fix the cooling pack so as to bring the buffer layer into contact with skin of the human body.

This therapeutic tool includes a cooling pack and a fixing unit as described here. Therefore, the therapeutic tool can be effectively used in cooling therapy.

The present international application claims priority to Japanese Patent Application, Tokugan, No. 2016-091593, filed on Apr. 28, 2016 and Japanese patent application, Tokugan, No. 2016-227282, filed on Nov. 22, 2016, the entire contents of which are incorporated herein by reference.

### REFERENCE SIGNS LIST

1 Cooling Pack
3 Cold Storage Layer
5 Buffer Layer
10 Cooling Pack
30 Cold Storage Layer
30a Freezing Medium
30b Cold-storage-layer-packaging Member
30c Articulation Mechanism
31 Cold Storage Layer
32 Cold Storage Layer
40 Cold Storage Layer
40a Freezing Medium
40b Cod-storage-layer-packaging Member
50 Buffer Layer
50a Antifreeze Medium
50b Buffer-layer-packaging Member
51 Buffer Layer
55 Cooling Pack
60 Cooling Pack
70 Cooling Pack
71 Cold Storage Layer
72 Buffer Layer
73 Cooling Pack
74 Cooling Pack
75 Cooling Pack
76 Pipe
77 Open/close Section
80 Thermal Manikin
82 Thermographic Camera
83 Cooling Pack (Rucksack-shaped Model)
84 Cooling Pack (Slipper-shaped Model)
85a Cooling Pack (Pillow-shaped Model)
85b Cover
86 Cooling Pack (Cap-shaped Model)
90 Deep-drawing Container
92 Filling Device
94 Lid-member-film Roller
96 Lid Member
100 Jig

1. A cooling pack that cools a human body, the cooling pack comprising:
a freezing medium having a phase transition temperature specified in a range of temperature, the range being suitable to maintain the human body at low temperature; and
a first container section containing the freezing medium therein,
wherein the freezing medium and the first container section constitute a cold storage layer.

2. The cooling pack according to claim 1, further comprising:
an antifreeze medium flexible at the phase transition temperature of the freezing medium; and
a second container section comprising a flexible material and containing the antifreeze medium therein,
wherein:
the antifreeze medium and the second container section constitute a buffer layer having a prescribed thermal conductivity, a prescribed thermal diffusivity, a prescribed thermal effusivity, and a prescribed heat transmission coefficient; and
the buffer layer transfers heat between the human body and the cold storage layer when brought into contact with skin of the human body.

3. The cooling pack according to claim 1, maintaining a site in contact with the skin of the human body at or above 17° C.

4. The cooling pack according to claim 2, wherein regarding the buffer layer, the thermal conductivity is from 0.584 to 0.590, the thermal diffusivity is from 1.503×10⁻⁷ to 1.537×10⁻⁷, the thermal effusivity is from 1,495 to 1,503, and the heat transmission coefficient is from 113 to 115.

5. The cooling pack according to claim 1, wherein the cold storage layer comprises: a plurality of first container sections; and an articulation mechanism configured to couple the plurality of first container sections.

6. The cooling pack according to claim 2, wherein the antifreeze medium is held by a part of the first container section and a part of the second container section.

7. The cooling pack according to claim 2, wherein the cold storage layer, as well as the antifreeze medium, is encased in the second container section.

8. The cooling pack according to claim 2, wherein the cold storage layer comprises a thermal insulation layer on a side thereof opposite from the buffer layer.

9. The cooling pack according to claim 1, wherein the cold storage layer comprises: a plurality of first container sections arranged in a matrix on a plane; and a plurality of articulation mechanisms configured to couple the plurality of first container sections.

10. The cooling pack according to claim 5, wherein the or each articulation mechanism is configured to be freely bendable and stretchable so that the plurality of first container sections is stacked by bending the or each articulation mechanism and expanded on a plane by stretching the or each articulation mechanism.

11. The cooling pack according to claim 1, further comprising:
a mitten section comprising a flexible material and shaped like a bag to contain a hand of the human body therein;
an antifreeze medium flexible at the phase transition temperature of the freezing medium; and
an antifreeze-medium-holding section provided in a thickness direction of the mitten section to hold the antifreeze medium,
wherein:
the mitten section, the antifreeze medium, and the antifreeze-medium-holding section constitute a buffer layer; and
the buffer layer transfers heat between the human body and the cold storage layer when brought into contact with skin of the human body.

12. The cooling pack according to claim 11, further comprising:
an open/close section configured to seal the mitten section; and
a pipe configured to couple to a depressurizing device configured to reduce pressure in the mitten section.

13. A therapeutic tool for use in cooling therapy, the therapeutic tool comprising:
a cooling pack according to claim 1; and
a fixing unit configured to fix the cooling pack so as to bring the cooling pack into contact with skin of the human body.
